(19) United States
(12) Patent Application Publication
(10) Pub. No.: US 2020/0239412 A1
(43) Pub. Date: Jul. 30, 2020

(54) NEW DERIVATES OF DHAA WITH ELECTROSTATIC TUNING

(71) Applicants: Fredrik ELINDER, Hässelby (SE); Xiongyu WU, Linköping (SE); Nina OTTOSSON, Linghem (SE); Peter KONRADSSON, Skänninge (SE); Malin SILVERÅ EJNEBY, Linköping (SE)

(72) Inventors: Fredrik ELINDER, Hässelby (SE); Xiongyu WU, Linköping (SE); Nina OTTOSSON, Linghem (SE); Peter KONRADSSON, Skänninge (SE); Malin SILVERÅ EJNEBY, Linköping (SE)

(21) Appl. No.: 16/651,260

(22) PCT Filed: Sep. 27, 2018
(86) PCT No.: PCT/SE2018/050992

§ 371 Date: Mar. 26, 2020

(30) Foreign Application Priority Data
Sep. 29, 2017 (SE) 1751207-0

(51) Int. Cl. C07C 305/18; C07F 9/09; C07C 309/14; C07C 309/04; C07C 233/63; C07C 233/62; C07C 57/50

(52) U.S. Cl. CPC C07C 305/18; C07F 9/09; C07C 309/14; C07C 57/50; C07C 233/63; C07C 233/62; C07C 309/04

(57) ABSTRACT

Dehydroabietic acid derivatives according to Formula Ia or Formula Ib and all stereoisomers thereof, having a linker chain A of 1 to 10 atoms selected from carbon, nitrogen and oxygen to a group X capable of being negatively charged at a physiological pH and covalently attached to the linker chain A, selected from carboxyl, sulfate, sulfonate and phosphate groups. The Dehydroabietic acid derivatives are useful for therapeutic treatment of hyperexcitability diseases

### BACKGROUND OF THE INVENTION

Voltage-gated ion channels play vital roles in generating cellular excitability, causing diseases when mutated, and being the target for drugs against diseases with increased cellular excitability, such as epilepsy, cardiac arrhythmia, and pain. Despite long-term efforts to develop effective medical drugs, many patients do not respond satisfactorily to the present-day drugs. For instance, one third of the patients with epilepsy do not respond properly. Therefore there is a need for new treatments. Voltage-gated ion channels, responsible for the generation and propagation of nervous and cardiac action potentials, are obvious targets.

Voltage-gated ion channels have a common structure: Four subunits packed together around an ion conducting pore. Each subunit has 6 transmembrane segments, named S1 to S6. The pore domain (S5-S6) includes the ion-conducting pore with the selectivity filter and the gates that open and close the pore. The voltage-sensor domain (VSD, S1-54) includes the positively charged voltage sensor S4 which moves through the channel protein during activation of the channel. Many of the present-day drugs block voltage-gated ion channels by plugging the ion-conducting pore. In most cases Na channels are targeted, but also Ca and K channels. However, there is an alternative mechanism that potentially can affect ion channel conductance—instead of blocking the ion conducting pore, a drug can affect (i) the gate that open and close the channel, or (ii) the voltage sensor that affects the gate. Retigabine, a new antiepileptic drug, opens the M-type K channel by acting on the gate and consequently shutting down electrical excitability. Spider toxins and some other compounds have been shown to specifically act on the voltage-sensor domain (VSD) of the ion channel, but there is presently no medical drug acting on the VSD.

A mechanism has been described, whereby charged hydrophobic compounds bind close to the VSD and thereby electrostatically affect the charged voltage sensor in the VSD. Negatively charged lipophilic substances (e.g. polyunsaturated fatty acids, PUFAs) was disclosed to bind to the lipid bilayer close to the ion channel and thereby shifting the channel's voltage dependence by electrostatically affecting the channel's voltage sensor, see Borjesson, S. I., et al Biophys. J. 95, 2242-2253 (2008). The binding site of PUFA is at the extracellular end of S3 and S4, distinct from previously described binding sites, and it is mainly the final opening step of the channel that is affected, see Borjesson, S. I. & Elinder, F., J. Gen. Physiol. 137, 563-640 (2011). However, to develop drug-like small-molecule compounds acting on voltage-gated channels with beneficial effects on for example epilepsy and pain, there is a need for other molecules than PUFAs.

A specific K channel is made supersensitive to PUFAs by inserting two extra positively charged residues in the extracellular end of the voltage sensor S4 (the 3R mutation), see. Ottosson, N. E. et al. J. Gen. Physiol. 143, 173-182 (2014). During certain circumstances, this channel increases the gain in open probability caused by PUFAs by more than 500 times compared to wild type. It was also described in this article that a resin acid, pimaric acid, had similar effects as PUFAs on the channel's voltage dependence.

Y-M Cui et al. Bioorg Med Chem, 18, 8642-8659 (2010) discloses that pimaric acid and other diterpene analogues, such as abietic acid and derivatives thereof have activity to open the calcium-activated BK channels, a subtype of K channels.

WO 2016/114707 discloses derivatives of dehydroabietic acid (DHAA) that are demonstrated as potent openers of a specific voltage-gated K channel and thereby exhibit usefulness as candidate drugs against cardiac arrhythmia and other hyperexcitability diseases including epilepsy and pain. The derivatization of Dehydroabietic acid on rings B and C suggested in WO 2016/114707 may enhance an anchoring capacity to the VSD, but does not further consider any electrostatic mechanisms of such compounds when opening voltage-gated K channels.

Various derivatives of dehydroabietic acid have also previously been disclosed as surfactants or antimicrobial agents, see for example WO 2016/051013; CN101972614; and JP61212547.

It is evident that there is a need for small-molecule drug candidates with improved properties to electrostatically open K channels and thereby being candidates to treat cardiac arrhythmia, epilepsy, and pain by compounds acting extracellularly on the voltage-sensor domain, rather than the traditional target, the ion-conducting pore domain.

### DESCRIPTION AND SUMMARY OF THE INVENTION

The present invention relates to dehydroabietic acid derivatives according to Formula Ia or Formula Ib and all stereoisomers thereof, wherein R₁₁, R₁₂, and R₁₄ are independently selected from hydrogen, halogen and R₂; R₁₃ is selected from hydrogen, halogen and R₃; and R₇ is selected from hydrogen, halogen, hydroxyl, carbonyl, and ═N—O—R₁; where R₁ is selected from hydrogen, and saturated or unsaturated lower alkyl groups selected from C₁-C₆ alkyl and C₂-C₆ alkenyl groups; R₂ and R₃ are independently from each other selected from straight, branched or cyclic saturated or unsaturated hydrocarbons comprising from 1 to 6 carbon atoms; wherein Formula Ia and Formula Ib are

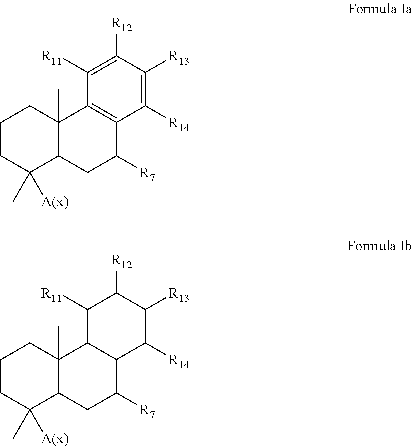

and wherein,

A(x) is defined as A-X and comprises a saturated, unsaturated, branched, unbranched, substituted or unsubstituted linker chain A of 1 to 10 atoms selected from carbon, nitrogen and oxygen, between the fused tricyclic moieties of Formulas Ia and Ib and at least one group X capable of being negatively charged at a physiological pH, selected from carboxyl, sulfate, sulfonate and phosphate groups.

The compounds of the present invention are preferably for use in the treatment in a hyperexcitability disease which is a condition of including increased excitability, higher than in normal cells. A smaller current is needed to be injected into the cell to cause an action potential. Preferably, the hyperexcitability diseases are epilepsy, cardiac arrhythmia, multiple sclerosis and pain.

The structures of Formula Ia and Formula Ib and defined above are generally termed “dehydroabietic acid derivatives”, however, in accordance with the present invention, for example the ring system in Formula Ib can also include one or several double bounds and thereby obtaining a ring system similar to abietic acid, pimaric acid or isopimaric acid. For this reason, compounds according to the present invention can validly be termed derivatives of one of abietic acid, pimaric acid, isopimaric acid or podocarpic acid.

In the following general part of the description of the invention general aspects of the invention are presented also in combination with embodiments of the invention as well as particular derivatives.

In one aspect, the derivatives are selected so that R₇, R₁₁, R₁₂, and R₁₄ are hydrogen R₁₃ is isopropyl.

In one aspect, the derivatives are selected so that the linker chain A is a carbon chain optionally interrupted by one or more atoms selected from nitrogen and oxygen and substituted with one or more of oxo groups, carboxyl groups, lower alkyl groups and halogen groups.

In one aspect, the derivatives are selected so that the linker chain A has 1 or 2 carbon atoms and X is a terminal carboxyl group. Particularly, according to this aspect, the derivatives are selected from 2-((1S,4aS)-7-isopropyl-1,4a-dimethyl-1,2,3,4,4a,9,10,10a-octahydrophenanthren-1-yl)acetic acid (Wu180) and 3-((1S,4aS)-7-isopropyl-1,4a-dimethyl-1,2,3,4,4a,9,10,10a-octahydrophenanthren-1-yl)propanoic acid (Wu179).

In one aspect, the derivatives are selected so that the linker chain A is a carbon chain, optionally interrupted with a nitrogen or oxygen atom, substituted with at least one of an oxo group and a carboxyl group, and wherein X is a terminal carboxyl group A particular derivative according to this aspect, is ((1R,4aS,10aR)-7-isopropyl-1,4a-dimethyl-1,2,3,4,4a,9,10,10a-octahydrophenanthrene-1-carbonyl)-L-aspartic acid (Wu148).

In one aspect, the derivatives are selected so that the linker chain A is a carbon chain comprising 2 to 10 atoms, of which at least one atom is nitrogen and X is a terminal carboxyl group. Particularly, according to this aspect, the derivatives are selected from ((1R,4aS,10aR)-7-isopropyl-1,4a-dimethyl-1,2,3,4,4a,9,10,10a-octahydrophenanthrene-1-carbonyl)glycine (Wu117); 3-((1R,4aS,10aR)-7-isopropyl-1,4a-dimethyl-1,2,3,4,4a,9,10,10a-octahydrophenanthrene-1-carboxamido)propanoic acid (Wu152); 4-((1R,4aS,10aR)-7-isopropyl-1,4a-dimethyl-1,2,3,4,4a,9,10,10a-octahydrophenanthrene-1-carboxamido)butanoic acid (Wu149); 3-(3-((1R,4aS,10aR)-7-isopropyl-1,4a-dimethyl-1,2,3,4,4a,9,10,10a-octahydrophenanthrene-1-carboxamido)propanamido)propanoic acid (Wu153); 4-((1R,4aR,4bR,10aR)-7-isopropyl-1,4a-dimethyl-1,2,3,4,4a,4b,10,10a-decahydrophenanthrene-1-carboxamido)butanoic acid (Wu157); and 4-(4-((1R,4aS,10aR)-7-isopropyl-1,4a-dimethyl-1,2,3,4,4a,9,10,10a-octahydrophenanthrene-1-carboxamido)butanamido)butanoic acid (Wu151).

In one aspect, the derivatives are selected so that the linker chain A comprises 2 to 5 atoms of which one optionally is nitrogen or oxygen and X is a terminal phosphate, sulfate or sulfonate group. Particularly, according to this aspect, the derivatives are selected from 1R,4aS,10aR)-7-isopropyl-1,4a-dimethyl-1,2,3,4,4a,9,10,10a-octahydrophenanthren-1-yl)methyl hydrogen sulfate (Wu161); 2-((1R,4aS,10aR)-7-isopropyl-1,4a-dimethyl-1,2,3,4,4a,9,10,10a-octahydrophenanthrene-1-carboxamido)ethane-1-sulfonic acid (Wu154); 3-((1R,4aS,10aR)-7-isopropyl-1,4a-dimethyl-1,2,3,4,4a,9,10,10a-octahydrophenanthrene-1-carboxamido)propane-1-sulfonic acid (Wu150); and ((1R,4aS,10aR)-7-isopropyl-1,4a-dimethyl-w 1,2,3,4,4a,9,10,10a-octahydrophenanthren-1-yl)methyl dihydrogen phosphate (Wu162).

The so described compounds can be substituted in positions R₇, R₁₁, R₁₂, R₁₃ and R₁₄ in three-ring system depicted in Formula Ia and Ib according to the following aspects and thereby combined with any of the described linker groups and associated charged groups X.

In one aspect, the groups R₂ and R₃ when defined as C1-C6 alkyl, C2-C6 alkenyl and C3-C6 cycloalkyl, as possible substitutes in positions R₁₁, R₁₂, R₁₄ and R₁₃ of formula Ia and Ib, hydrogens of said alkyl, alkenyl, and cycloalkyl groups optionally can be substituted with at least one halogen.

In one aspect the derivatives R₃ is an isopropyl group. In this aspect, R₁₃ preferably is isopropyl.

In one aspect, the dehydroabietic acid derivatives according to the invention are selected from compounds substituted according to groups a) to m) wherein:
a) R₁₂ is —F, R₁₁ and R₁₄ is —H, R₃ is isopropyl, and R₇ is selected from —H, ═O and ═N—O—R₁, where R₁ is selected from —H, —CH₃, and —CH₂—CH═CH₂;
b) R₁₁ and R₁₂ is —H, R₁₄ is —F, R₃ is isopropyl, and R₇ is selected from —H, ═O and ═N—O—R₁, where R₁ is selected from —H, —CH₃, and —CH₂—CH═CH₂;
c) R₁₂ is —Cl, R₁₁ and R₁₄ is —H, R₃ is isopropyl, and R₇ is selected from —H, ═O and ═N—O—R₁, where R₁ is selected from —H, —CH₃, and —CH₂—CH═CH₂;
d) R₁₁ and R₁₂ is —H, R₃ is isopropyl, R₁₄ is —Cl, and R₇ is selected from —H, ═O and ═N—O—R₁, where R₁ is selected from —H, —CH₃, and —CH₂—CH═CH₂;
e) R₁₁ is —Cl, R₃ is isopropyl, and R₇ and R₇ is selected from —H, ═O and ═N—O—R₁, where R₁ is selected from —H, —CH₃, and —CH₂—CH═CH₂, R₁₂ and R₁₄ are —H;
f) R₁₁ and R₁₄ are —Cl, R₁₂ is —H, R₃ is isopropyl and R₇ is selected from —H, ═O and ═N—O—R₁, where R₁ is selected from —H, —CH₃, and —CH₂—CH═CH₂;
g) R₁₁ and R₁₄ is —H, R₁₂ is —Br, R₃ is isopropyl, and R₇ is selected from —H, ═O, ═N—O—R₁, where R₁ is selected from —H, —CH₃, and —CH₂—CH═CH₂;
h) R₁₁ and R₁₂ is —H, R₃ is isopropyl, R₁₄ is —Br, and R₇ is selected from —H, ═O and ═N—O—R₁, where R₁ is selected from —H, —CH₃, and —CH₂—CH═CH₂;
i) R₁₂ is —I, R₁₁ and R₁₄ is —H, R₃ is isopropyl, and R₇ is selected from —H, ═O and ═N—O—R₁, where R₁ is selected from —H, —CH₃, and —CH₂—CH═CH₂;
j) R₁₁ and R₁₂ is —H, R₁₄ is —I, R₃ is isopropyl, and R₇ is selected from —H, ═O and ═N—O—R₁, where R₁ is selected from —H, —CH₃, and —CH₂—CH═CH₂;
k) R₁₁ is —H, R₁₂ and R₁₄ is —Cl, R₃ is isopropyl, and R₇ is selected from —H, ═O and ═N—O—R₁, where R₁ is selected from —H, —CH₃, and —CH₂—CH═CH₂;
l) R₁₁, R₁₂ and R₁₄ is —Cl, R₃ is isopropyl, and R₇ is selected from —H, ═O, and ═N—O—R₁, where R₁ is selected from —H, —CH₃, and —CH₂—CH═CH₂; and
m) R₁₁, R₁₂ and R₁₄ is —H, R₃ is isopropyl, and R₇ is selected from —H, ═O, —OH, and ═N—O—R₁, where R₁ is selected from —H, —CH₃, and —CH₂—CH═CH₂.

In one aspect the dehydroabietic acid derivatives are selected so that R₁₁, R₁₂, and R₁₄ are independently selected from hydrogen and halogen, R₃ is isopropyl; and R₇ is —H, ═N—O—CH₃ or ═N—O—CH₂—CH═CH₂.

In one aspect, the dehydroabietic acid derivatives are selected so that R₇ is —H, ═N—O—CH₃ or ═N—O—CH₂—CH═CH₂; R₃ is isopropyl; and R₁₁, R₁₂ and R₁₄ independently are selected from —H, —F, —Cl, —Br.

In one aspect of the invention the dehydroabietic acid derivatives are selected so that R₇ is ═O, ═N—O—CH₃ or ═N—O—CH₂—CH═CH₂; R₃ is isopropyl; and R₁₁, R₁₂ and R₁₄ independently are selected from—hydrogen and halogen with the proviso that R₁₂ is not bromo. In one embodiment of this aspect the halogen is iodo, preferably R₁₂ is iodo.

According to one aspect, the dehydroabietic acid derivatives are selected so R₇ is selected from hydrogen, halogen, and ═N—O—R₁ and where R₁₃ is selected from H or halogen. In one embodiment of this aspect the halogen is chloro. Preferably according to this aspect, the linker chain A is one carbon atom and X is sulfate. A particular derivative according to this aspect is ((1R,4aS,10aR)-6,7,8-trichloro-1,4a-dimethyl-2,3,4,9,10,10a-hexahydrophenanthrene-1-yl)methyl hydrogen sulfate (Wu181).

According to one aspect, the dehydroabietic acid derivatives are selected so R₁₁ and R₁₄ are hydrogen, R₁₂ is R₂, R₁₃ is R₃, and R₇═N—O—R₁.

According to one aspect, the dehydroabietic acid derivatives are selected so R₁₁ is hydrogen, R₁₂ and R₁₄ is selected from hydrogen and halogen R₃ is isopropyl, and R₇ is carbonyl.

According to one aspect, the dehydroabietic acid derivatives are used as defined above, wherein R₁₁ is hydrogen, R₁₂ and R₁₄ is selected from hydrogen and —I, R₃ is isopropyl, and R₇ is carbonyl or ═N—OH.

According to one aspect, the compounds of the invention are operable on the voltage-gated Kv (potassium) channel of the Kv family is selected from at least one of the subfamilies Kv-1, Kv-2, Kv-3, Kv-4, and Kv-7.

According to another aspect, the compounds which are operable voltage-gated potassium (Kv) channel of the Kv family are operable on the subfamily Kv-1 or Kv-7.

In one aspect one or more the previously described dehydroabietic acid derivatives are used in treatment, i.e. methods of treatment of epilepsy or pain. At least one dehydroabietic acid derivative according to the invention is administered in a therapeutically active amount in a pharmaceutical dose form.

In one aspect the previously defined dehydroabietic acid derivatives are for use in treatment i.e. methods of treatment of cardiac arrhythmia, especially atrial fibrillation (AF) in cardiac arrhythmia. At least one dehydroabietic acid derivative according to the invention is administered in a therapeutically active amount in a pharmaceutical dose form.

The present invention accordingly extends to pharmaceutical compositions comprising one or more of the previously described derivatives.

### DETAILED AND EXPERIMENTAL DESCRIPTION OF THE INVENTION

For the definition of the following terms, see WO 2016/114707.
An ion channel
A voltage-gated ion channel or a voltage-gated channel
A voltage-gated K ion channel (Kv channel)
Kv1, Kv2, Kv3, Kv4, and Kv7
A Shaker K channel
A voltage-sensor domain (VSD)
Opening of a Kv channel
Extracellular activator
Affecting or shifting the voltage dependence of voltage-gated Kv channel
Potency or “a potent shifter”
Hyperpolarization
Excitability
Hyperexcitability
Hypoexcitability

In the context of the present invention, both when it is described in the previous generalized aspects and in the detailed forms in the following experimental part, the additional following definitions can be used.

A K channel is an ion channel selective for potassium ions.

A Na channel is an ion channel selective for sodium ions.

A Ca channel is an ion channel selective for calcium ions.

A Kv7.2/7.3 channel (hereinafter called M-channel) is a heteromeric Kv channel that belongs to the Kv7 subfamily.

A calcium-activated BK channel (hereinafter called BK channel) is a K channel that both is activated by voltage and the intracellular concentration of calcium.

The Voltage sensor S4 has a number of positively charged amino acids that detects and moves as a response to changes in membrane potential. The movement of S4 causes the opening and closing of a voltage-gated ion channel.

A gating charge refers to the positively charged amino acids in the voltage sensor S4.

A Polyunsaturated fatty acid (PUFA) is a molecule with a lipophilic part and a carboxyl group that makes enables it to be negatively charged.

Docosahexaenoic acid (DHA) is a PUFA.

Steady-state current refers to the invariant current recorded when clamping the membrane long enough.

Tail current refers to the current recorded when the voltage is switched from a level where the channel is at least partly open to a new level.

Anchor refers to the three-ring motif, the lipophilic part, of the resin acid or derivative thereof.

Effector refers to the chemical group attached to the anchor, generally containing the charge of the resin acid or derivative thereof. The effector refers to the at least one group X capable of being negatively charged at a physiological pH, selected from carboxyl, sulfate, sulfonate and phosphate groups

Stalk refers to the linker chain of atoms between C4 at the anchor and the effector. Linker chain and stalk are used with the same meaning in the present document in different context and refers to the chain of atoms between the fused tricyclic moieties of Formulas Ia and Ib a negatively charged group X, as previously defined.

Stalk lengths refers to number of atoms between the anchor and effector of the resin acid or derivative thereof.

Anchoring capacity refers to the capacity of a compound to bind close to the VSD.

Theoretical pKa refers to the logarithm of the acid dissociation constant calculated using a software according to Material and Methods

Theoretical pH dependence refers to calculated molecular microspecies (uncharged or with different valance) distribution at different pH

Functional pKa refers to the pKa calculated from recordings and corresponds to the pH where half of the maximum shift was achieved.

Functional pH dependence refers to G(V) shifting effects from recordings at different pH

Cut-off model refers to a model that based on electrostatic energy calculations predicts the preferred position of a charge in the effector.

Cut-off length refers to the stalk length were the G(V) shifting effect drastically change

Permanently charged refers to a compound with a theoretical pKa value below 1

Partially charged refers to a compound that have a functional pKa value close to 7, therefore both uncharged and charged at physiological pH

Materials and Methods

Molecular Biology and Expression of Channels

Experiments were made with the Shaker H4 channel incapable of fast N-type inactivation due to a Δ6-46 deletion, referred to as the wt Shaker Kv channel. The 3R Shaker Kv channel with two additional positively charged arginines (M356R/A359R) in the extracellular end of S4, was also used. The 3R Shaker Kv channel is more sensitive to the PUFA docosahexaenoic acid (DHA) and resin acids compared to the wt Shaker Kv channel.

Experiments were also made with the human hKv7.2/Kv7.3, referred to as the M-channel. cRNAs were mixed in a 1:1 molar ratio before injection into oocytes. hKv7.2 [GenBank Acc. No. NM_004518]. hKv7.3 [GenBank Acc. No NM_004519].

For the Shaker channels, Bluescript II KS(+) plasmid was linearized with HindIII and synthesis of cRNA were made with the T7 mMessage mMachine kit (Ambion, Austin, Tex.). Point mutations around S4, on the Shaker Kv channel, were introduced using QuickChange Site-Directed Mutagenesis Kit (Agilent Technologies, Santa Clara, Calif.) and sequencing were used for verification.

50 nl cRNA was injected into Xenopus laevis oocytes using a Nanoject injector (Drummond Scientific, Broomall, Pa.) and the oocytes were stored at 8° C. in a modified Barth's solution (MBS) before experiments (16° C., M-channel, 1-2 days). The MBS contained (in mM): 88 NaCl, 1 KCl, 2.4 NaHCO₃, 15 HEPES, 0.33 Ca(NO3)2, 0.41 CaCl2), and 0.82 MgSO4, pH was adjusted to 7.5 by NaOH and the MBS was supplemented sodium pyruvate (2.5 mM). All animal experiments were approved by the Linkoping's local Animal Care and Use Committee.

Electrophysiology

Currents were measured 1-6 days after cRNA injection using the two-electrode voltage clamp technique (GeneClamp 500B amplifier, Axon instruments). Signals were low pass filtered at 5 kHz and digitized by a Digidata 1440A converter (Molecular Devices, Sunnyvale, Calif.). The amplifiers leak compensation was used and Clampex 10.2 software (Molecular Devices) was used to obtain data and create voltage protocols. The extracellular control solution contained (in mM): 1 KCl, 88 NaCl, 0.8 MgCl2, 0.4 CaCl2) and 15 HEPES. pH were adjusted with NaOH. Electrode pipettes were made from borosilicate glass capillaries (Harvard apparatus, Kent, UK) using a two stage electrode puller (Narishige, Tokyo, Japan) and resistance was between 0.5-2.0 MO once filled with 3 M KCl solution. In the voltage protocol for the wt and 3R Shaker channel, holding potential was set to −80 mV and steady-state currents were measured at voltages between −80 and +50 mV (wt Shaker Kv channel) or +70 mV (3R Shaker Kv channel), using 5 mV increments, 100 ms. The S4 mutants of the Shaker Kv channel has previously been characterized, and voltage protocols were set accordingly, see Ottosson, N. E. et al. J. Gen. Physiol. 143, 173-182 (2014). For the M-channel, holding potential was set to −100 mV, test-voltages were measured between −120 and +50 mV (10 mV increments for 2 s) and tail currents were measured at −30 mV.

Control solution was perfused to the recording chamber (0.5 ml/min) and the test solution was added manually with a syringe, in a volume large enough to replace the control solution manifold. Resin acids compounds were dissolved in DMSO (100 mM) and stored at −20° C. Just prior to experiment the compound was diluted to desired concentration in control solution. Recovery was measured after continuously perfusion of control solution, maximum 5 min. All recordings were made with the perfusion off. Experiments were carried out in room temperature (20-23° C.) and chemicals were purchased from Sigma Aldrich if not stated otherwise.

Compound Synthesis and Calculated Chemical Properties

Marvin was used for drawing chemical structures (Marvin 16.12.9, 2016, ChemAxon (http://www.chemaxon.com). The logarithm of the acid dissociation constant, pKa, for the ionic forms of the compounds were calculated using the Marvin Calculation Plugin. The pH range was set between −2 and 16, temperature to 298 Kelvin, and the pKa was obtained from the global mass and charge conservation law (Macro mode). The ionic strength was considered to be 0.1 mol/L. Octanol-Water partitioning coefficient, log P, for uncharged compounds were calculated using Marvin Calculation Plugin. The consensus model in Chemaxon was used for calculations and electrolyte concentrations was set to 0.1 mol/dm3 for Cl⁻, Na⁺ and K⁺. Calculated pKa and log P values for all compounds are listed in Table 1.

Analysis

Currents were leak subtracted and steady-state currents were measured 80-86 ms after onset of the test voltages (Clampfit 10.5, Molecular Devices). K conductance, G_{K}, was calculated using,

G_{K}(V)=I_{K}/(V−V_{K}), (1)

where I_{K} is the steady-state current, V the absolute membrane voltage and V_{K} the reversal potential for K ions (−80 mV), and the following Boltzmann curve was used to fit the data:

G_{K}(V)=A/(1+exp((V_{1/2}−V)/s)ⁿ (2)

where A is the amplitude of the curve, V_{1/2} the midpoint when n=1, s the slope and n an exponent for better curve fitting, set to n=4. The voltage shift was calculated at 10% of maximal conductance in control curve.

For the M channel tail currents were measured 18 ms after onset and then plotted against the test voltage. The Boltzmann curve (Eq. 2) was used to fit data and the slope was set to shared value between control and test compound. V_{1/2} was used to calculate the G_{K}(V) shift.

To quantify the concentration dependence or pH dependence of the G_{K}(V) shifts the following equation was used:

ΔV_{G}(V)=ΔV_{MAX}/(1+c_{1/2}/c), (3)

where ΔV_{G}(v) is the voltage shift, ΔV_{MAX} is the amplitude of the curve, c the concentration, c_{1/2} the concentration at which half maximum response occur. For compounds with no pH dependence the data was fitted with a horizontal line, with the slope set to 0.

For fitting the decreased G(V) shift with increasing stalk length/carbon length at pH 7.4, a one phase exponential decay curve was used:

ΔV_{G(V)}=A*exp(−I/λ), (4)

where ΔV_{G(V)} is the voltage shift, A is the maximal amplitude of the curve where the stalk length is 0 atoms long, I is the stalk length (in number of atoms), and λ is the length constant (in number of atoms).

The pH dependence for Wu162 with valance −1 and −2 was fitted with the following equation:

ΔV=(min1−A)/(1+10\hat{ }(X−pKₐ1))−(min2−A))/(1+10\hat{ }(x−pKₐ2)) (5)

where min1 is the bottom for valence −2, min2 is the bottom for uncharged molecules set to 0, A is the maximal amplitude, pKₐ1 and pKₐ 2 is set to 6.89 and 1.87, respectively, derived from the theoretical pH dependence for molecule microspecies with valance −1 and −2 (FIG. 6C).

Cut-Off Model

The electrostatic energy for the simple model system (FIG. 4A) is calculated treating the two charges as point charges in a perfect semi-infinite dielectric medium (the lipid bilayer, ε₁=2) adjacent to another perfect semi-infinite dielectric medium (water, ε₂=80). We use the method of image charges to calculate the electrostatic energy of the model system, where the influence of the water part (medium 2) is replaced by an image charge (in the water) at a distance z_{image} from the interface opposite to the charge in question (i.e. d for the gating charge and |z| for the effector charge). The image charge given by:

q′ᵢ=−((ε₂−ε₁)/(ε₂+ε₁)qᵢ (6)

is treated as being present in the lipid layer (medium 1). With ε₁=2 and ε₂=80 the image charge is close to −qᵢ.

The electrostatic energy is calculated as:

Wₑ=½ΣqᵢVᵢ, (7)

where the sum is over the two charges qᵢ and Vi is the potential at qᵢ due to its image charge and the other charge and its image charge.

The simple model in FIG. 4A cannot be used close to the interface since then the change in self energy of the charges has to be taken into account (the self-energy is high in the lipid and low in the water). FIG. 4B is meant to indicate that at z=0 the total energy (including the self-energy) drops very fast to become very low in the water (the electrostatic contribution from the charges in the lipid becomes also very small in the water).

Statistics

Average values are expressed as Mean±SEM. A two-tailed one sample t-test were the mean value was compared to a hypothetical value of 0 was used to analyse G(V)-shifts. When comparing groups, one-way ANOVA with Bonferroni's multiple comparison tests or Dunnett's multiple comparison test was used. Correlation analysis was done with Pearson's correlation test and linear regression. p<0.05 was considered significant.

Synthesis of Compounds of the Invention

Supplementary Methods: Compounds Synthesis

General Methods and Materials

All the solvents and reagents were used without further distillation or drying. Dehydroabietic acid was bought from BOC Sciences, other reagents from Sigma-Aldrich. Analytical thin-layer chromatography was performed on the Merk silica gel 60F₂₅₄ glass-backed plates. Flash chromatography was performed with silica gel 60 (particles size (0.040-0.063 mm). Preparative LC was run on either a Gilson Unipoint system with a Gemini C18 column (100×21.20 mm, 5 micron) or a Waters system with a XSELECT Phenyl-Hexyl column (250×19 mm, 5 micron), under neutral condition using gradient CH₃CN/water as eluent (A, water phase: 95:5 water/CH₃CN, 10 mM NH₄OAc; B, organic phase: 90:10 CH₃CN/water, 10 mM NH₄OAc). NMR spectra were recorded on a Varian Avance 300 MHz with solvent indicated. Chemical shift was reported in ppm on the 5 scale and referenced to solvents peak (CDCl₃: δ_{H}=7.26 ppm, δ_{C}=77.16 ppm; Acetone-d6: δ_{H}=2.05 ppm, δ_{C}=29.84 ppm). ¹⁹F-NMR and ³¹P-NMR were recorded using benzotrifluoride in CDCl₃ (δ_{F}=−63.24 ppm) and 85% H₃PO₄ in D₂O (δ_{P}=0 ppm) as external standards respectively.

General Procedure of Amide Coupling

To dehydroabietic acid (70 mg, 0.233 mmol) and DiPEA (66.3 mg, 0.513 mmol) in 7 mL acetonitrile was added TBTU (78.6 mg, 0.245 mmol) and stirred at rt for about 0.5 h, then amino acid or ester (0.280 mmol) was added at rt and stirred over two nights. The solution was concentrated and dissolved in 25 mL DCM, washed with 15 mL water, concentrated and purified on flash silica chromatography or preparative LC to give the desired product.

Synthesis of Wu109-Wu111

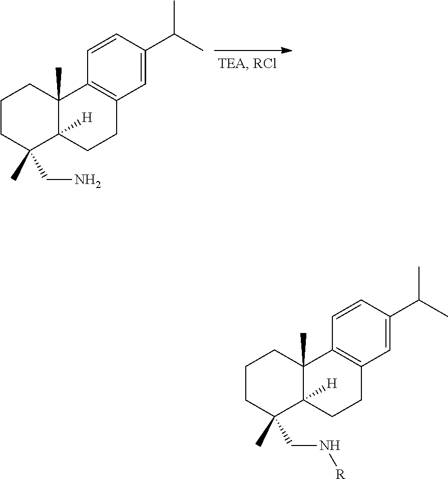

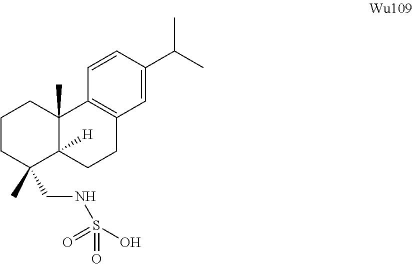

### (((1R,4aS,10aR)-7-isopropyl-1,4a-dimethyl-1,2,3,4,4a,9,10,10a-octahydrophenanthren-1-yl)methyl)sulfamic Acid (Wu109)

To the solution of dehydroabietyl amine (60% technical purity) (66 mg, 0.231 mmol) and triethylamine (174.9 mg, 1.73 mmol) in DCM (3 mL) added chlorosulfonic acid (53.2 mg, 0.457 mmol). The mixture was stirred at rt for 3 h. Concentrated and purified using preparative LC (B/A: 20:80 to 90:10). The desired fractions was diluted with water, acidified with 2N HCl to pH˜1, extracted with EtOAc (10 mL×2), concentrated to give Wu109 (56.6 mg, 67%). ¹HNMR (CDCl₃, 300 MHz) δ 7.16 (d, J=8.1 Hz, 1H), 7.01 (dd, J=8.1, 1.8 Hz, 1H), 6.90 (d, J=1.8 Hz, 1H), 3.46 (d, J=12.3 Hz, 1H), 3.07 (d, J=12.1 Hz, 1H), 2.98-2.76 (m, 3H), 2.34 (br d, J=12.9 Hz, 1H), 1.87-1.66 (m, 4H), 1.60 (br d, J=12.9 Hz, 1H), 1.47-1.27 (m, 3H), 1.24 (s, 3H), 1.23 (d, J=7.2 Hz, 6H), 1.05 (s, 3H). ¹³CNMR (CDCl₃, 75 MHz) δ 146.3, 146.0, 134.2, 127.0, 124.2, 124.1, 57.5, 47.5, 37.9, 37.6, 36.3, 35.8, 33.6, 29.7, 25.3, 24.1, 24.0, 19.3, 18.5, 17.4. MS (ESI⁻): m/z calcd for C₂₀H₃₀NO₃S (M-H⁻) 364.19, found 364.26.

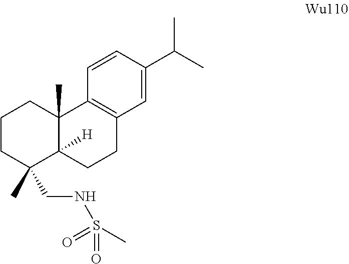

### N-(((1R,4aS,10aR)-7-isopropyl-1,4a-dimethyl-1,2,3,4,4a,9,10,10a-octahydrophenanthren-1-yl)methyl)methanesulfonamide (Wu110)

Following the synthesis procedure of Wu109, Wu110 was synthesized using dehydroabietyl amine (60% technical purity) (130 mg, 0.455 mmol), triethylamine (92.1 mg, 0.91 mmol) and methylsulfonyl chloride (57.4 mg, 0.501 mmol) as starting materials in 70% yield. ¹HNMR (CDCl₃, 300 MHz) δ 7.17 (d, J=8.4 Hz, 1H), 6.99 (dd, J=8.4, 1.8 Hz, 1H), 6.90 (d, J=1.8 Hz, 1H), 4.67 (t, J=6.9 Hz, 1H), 3.05-2.77 (m, 8H), 2.30 (br d, J=12.6 Hz, 1H), 1.82-1.64 (m, 4H), 1.52 (dd, J=13.8, 3.9 Hz, 1H), 1.47-1.27 (m, 3H), 1.23 (d, J=6.9 Hz, 6H), 1.22 (s, 3H), 0.96 (s, 3H). ¹³CNMR (CDCl₃, 75 MHz) δ 147.1, 145.8, 134.7, 126.9, 124.2, 123.9, 54.0, 45.0, 40.1, 38.4, 37.5, 37.1, 35.9, 33.6, 30.0, 25.3, 24.1, 24.08, 18.9, 18.62, 18.57. MS (ESI⁻): m/z calcd for C₂₁H₃₂NO₂S (M-H⁻) 362.22, found 362.29.

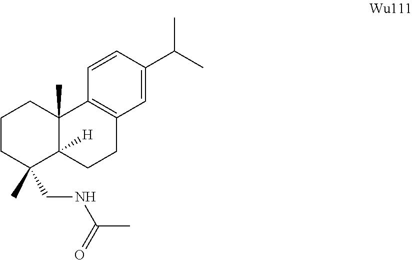

### N-(((1R,4aS,10aR)-7-isopropyl-1,4a-dimethyl-1,2,3,4,4a,9,10,10a-octahydrophenanthren-1-yl)methyl)acetamide (Wu111)

Following the synthesis procedure of Wu109, Wu111 was synthesized using dehydroabietyl amine (60% technical purity) (130 mg, 0.455 mmol), triethylamine (92.1 mg, 0.91 mmol) and acetyl chloride (39.3 mg, 0.501 mmol) as starting materials in 75% yield. ¹HNMR (CDCl₃, 300 MHz) δ 7.17 (d, J=8.1 Hz, 1H), 7.00 (dd, J=8.1, 1.8 Hz, 1H), 6.90 (d, J=1.8 Hz, 1H), 5.50 (br s, 1H), 3.24 (dd, J=13.8, 5.1 Hz, 1H), 3.09 (dd, J=13.5, 5.1 Hz, 1H), 2.98-2.74 (m, 3H), 2.29 (br d, J=12.6 Hz, 1H), 1.98 (s, 3H), 1.94-1.55 (m, 4H), 1.46-1.28 (m, 4H), 1.23 (d, J=6.9 Hz, 6H), 1.22 (s, 3H), 0.94 (s, 3H). ¹³CNMR (CDCl₃, 75 MHz) δ 170.4, 147.3, 145.8, 134.9, 127.1, 124.3, 124.0, 50.1, 45.3, 38.5, 37.6, 37.4, 36.3, 33.6, 30.3, 25.4, 24.13, 24.09, 23.7, 19.1, 18.9, 18.7. MS (ESI⁻): m/z calcd for C₂₂H₃₂NO (M-H⁻) 326.25, found 326.37.

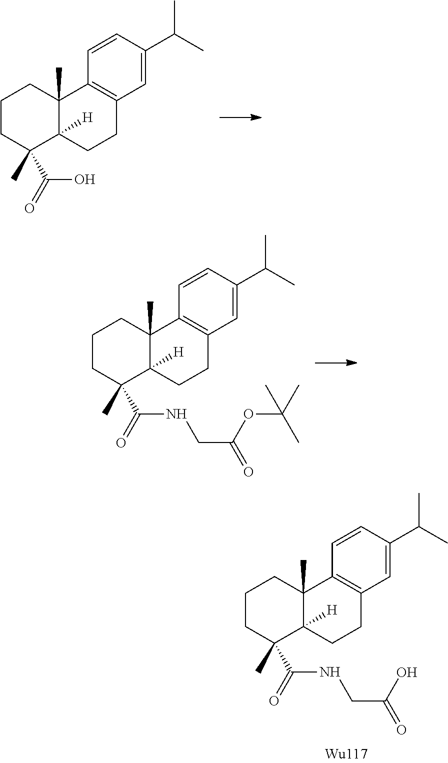

### ((1R,4aS,10aR)-7-isopropyl-1,4a-dimethyl-1,2,3,4,4a,9,10,10a-octahydrophenanthrene-1-carbonyl)glycine (Wu117)

Followed the general procedure of amide coupling, dehydroabietic acid (30 mg, 0.10 mmol) and glycin tert-buthyl ester hydrochloride (33.5 mg, 0.20 mmol) were used. The reaction mixture was stirred at rt for one night. The reaction mixture was filtered, concentrated and dissolve in DCM (2 mL) followed by adding of 500 uL TFA. The reaction mixture was stirred at rt overnight. The reaction mixture was concentrated and purified using silica gel chromatography with EtOAc/n-heptane/HCOOH (50:50:0.5 to 100:0:0.5) to give Wu117 (32.4 mg, 91%). ¹HNMR (CDCl₃, 300 MHz) δ 8.09 (br s, 1H), 7.14 (d, J=8.1 Hz, 1H), 6.98 (dd, J=8.1, 1.5 Hz, 1H), 6.86 (d, J=1.5 Hz, 1H), 6.64 (t, J=5.1 Hz, 1H), 4.06-3.87 (m, 2H), 2.89-2.75 (m, 3H), 2.28 (br d, J=12.6 Hz, 1H), 2.11 (dd, J=12.3, 1.5 Hz, 1H), 1.82-1.62 (m, 4H), 1.60-1.41 (m, 3H), 1.29 (s, 3H), 1.21 (d, J=6.6 Hz, 6H), 1.20 (s, 3H). ¹³CNMR (CDCl₃, 75 MHz) δ 180.1, 173.5, 147.0, 145.9, 134.8, 127.0, 124.1, 124.0, 47.4, 45.6, 42.3, 38.0, 37.2, 33.6, 30.0, 25.4, 24.1, 21.2, 18.8, 16.5. MS (ESI⁻): m/z calcd for C₂₂H₃₀NO₃ (M-H⁻) 356.22, found 356.31.

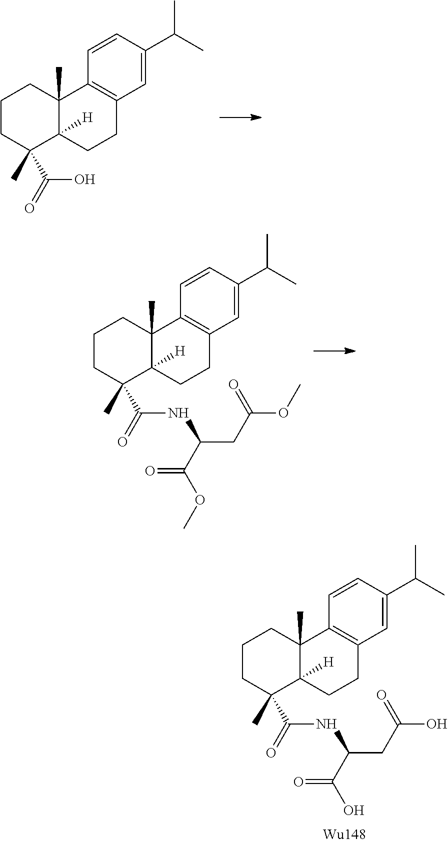

### ((1R,4aS,10aR)-7-isopropyl-1,4a-dimethyl-1,2,3,4,4a,9,10,10a-octahydrophenanthrene-1-carbonyl)-L-aspartic acid (Wu148)

Followed the general procedure of amide coupling, L-aspartic acid dimethylester hydrochloride (55.3 mg, 0.280 mmol) was used. The mixture was stirred at rt overnight, concentrated and purified using flash silica gel chromatography with EtOAc/n-heptane (25:75 to 50:50) to give the intermediate product of methyl ester (69.5 mg, 67%). ¹HNMR (CDCl₃, 300 MHz) δ 7.17 (d, J=8.4 Hz, 1H), 7.00 (dd, J=8.4, 1.8 Hz, 1H), 6.88 (d, J=1.8 Hz, 1H), 6.83 (br d, J=7.8 Hz, 1H), 4.87 (dt, J=7.8, 4.5 Hz, 1H), 3.76 (s, 3H), 3.70 (s, 3H), 3.02 (dd, J=16.8, 4.5 Hz, 1H), 2.94-2.77 (m, 4H), 2.32 (br d, J=12.9 Hz, 1H), 2.11 (dd, J=12.3, 2.1 Hz, 1H), 1.84-1.44 (m, 7H), 1.30 (s, 3H), 1.23 (s, 3H), 1.22 (d, J=6.9 Hz, 6H). ¹³CNMR (CDCl₃, 75 MHz) δ 178.3, 171.8, 171.6, 147.0, 145.8, 134.7, 127.0, 124.2, 124.0, 52.9, 52.1, 48.9, 47.4, 45.8, 38.1, 37.3, 37.2, 36.1, 33.6, 30.2, 25.5, 24.1, 21.2, 18.8, 16.5.

To the intermediate (69.5 mg, 0.156 mmol) added LiOH monohydrate (37.9 mg, 0.936 mmol) and THF/water (5 ml/2 mL). The mixture was stirred at rt for 2 h. The mixture was diluted with water (5 mL), adjusted pH to about 4, extracted with EtOAc (12 mL×3). The organic phase was concentrated, dissolved in DCM (DCM was used to remove insoluble substances.) and concentrated again to give Wu148 (64.5 mg, 99%). ¹HNMR (CDCl₃, 300 MHz) δ 9.19 (br s, 2H), 7.15 (d, J=8.1 Hz, 1H), 7.03 (br d, J=7.8 Hz, 1H), 6.99 (dd, J=8.1, 1.8 Hz, 1H), 6.87 (d, J=1.8 Hz, 1H), 4.94-4.82 (m, 1H), 3.50 (dd, J=17.1, 4.2 Hz, 1H), 2.91-2.74 (m, 4H), 2.30 (d, J=12.0 Hz, 1H), 2.12 (d, J=12.3 Hz, 1H), 1.86-1.40 (m, 7H), 1.29 (s, 3H), 1.22 (s, 3H), 1.21 (d, J=6.9 Hz, 6H). ¹³CNMR (CDCl₃, 75 MHz) δ 179.6, 175.9, 175.2, 146.9, 145.9, 134.7, 127.1, 124.2, 124.1, 48.9, 47.5, 45.6, 38.0, 37.1, 36.0, 33.6, 30.1, 25.5, 24.1, 21.3, 18.8, 16.4. MS (ESI⁻): m/z calcd for C₂₄H₃₁NO₅ (M-H⁻) 414.23, found 414.36.

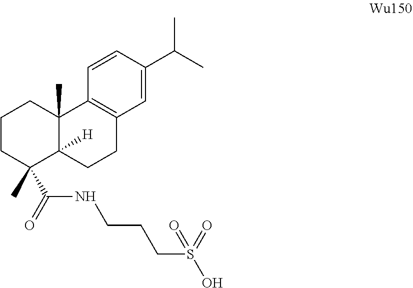

### 3-((1R,4aS,10aR)-7-isopropyl-1,4a-dimethyl-1,2,3,4,4a,9,10,10a-octahydrophenanthrene-1-carboxamido)propane-1-sulfonic acid (Wu150)

Followed the general procedure of amide coupling, the reaction mixture was purified using preparative LC (B/A: 10:90 to 80:20) to give Wu150 (34.4 mg, 35%). Wu150: ¹HNMR (CDCl₃, 300 MHz) δ 9.20 (br s, 1H), 8.06 (br s, 1H), 7.03 (d, J=8.4 Hz, 1H), 6.95 (dd, J=8.4, 1.5 Hz, 1H), 6.82 (d, J=1.5 Hz, 1H), 3.58-3.37 (m, 2H), 3.03-2.87 (m, 2H), 2.86-2.70 (m, 3H), 2.21-1.93 (m, 4H), 1.83-1.44 (m, 5H), 1.40-1.23 (m, 2H, not including methyl group), 1.27 (s, 3H), 1.19 (d, J=6.9 Hz, 6H), 1.13 (s, 3H). ¹³CNMR (CDCl₃, 75 MHz) δ 181.6, 146.7, 145.9, 134.4, 126.9, 124.2, 124.1, 49.0, 47.6, 44.8, 40.1, 37.7, 37.0, 36.5, 33.6, 29.9, 25.3, 24.3, 24.2, 21.3, 18.6, 16.5. MS (ESI⁻): m/z calcd for C₂₃H₃₄NO₄S (M-H⁻) 420.22, found 420.31.

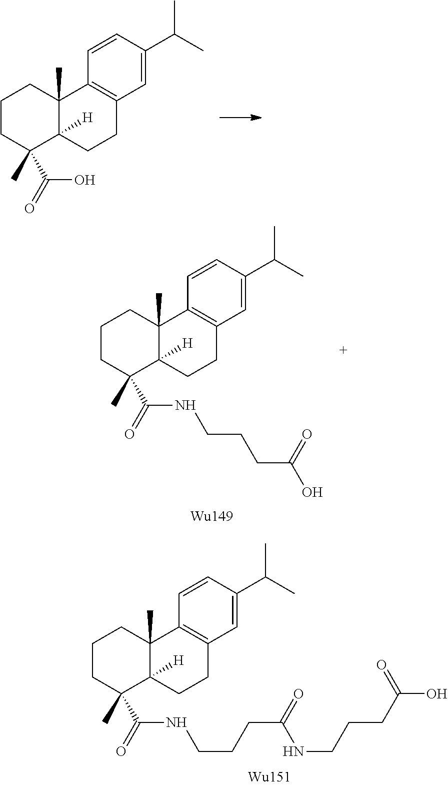

### 4-((1R,4aS,10aR)-7-isopropyl-1,4a-dimethyl-1,2,3,4,4a,9,10,10a-octahydrophenanthrene-1-carboxamido)butanoic Acid (Wu149) and 4-(4-((1R,4aS,10aR)-7-isopropyl-1,4a-dimethyl-1,2,3,4,4a,9,10,10a-octahydrophenanthrene-1-carboxamido)butanamido)butanoic Acid (Wu151)

Followed the general procedure of amide coupling, the reaction mixture was purified using preparative LC (B/A: 10:90 to 80:20) to give Wu151 (7.7 mg, 7%) and Wu149 (29.9 mg, 33%). Wu149: ¹HNMR (CDCl₃, 300 MHz) δ 7.15 (d, J=8.4 Hz, 1H), 6.99 (dd, J=8.4, 1.8 Hz, 1H), 6.87 (d, J=1.8 Hz, 1H), 6.16 (t, J=5.4 Hz, 1H), 3.39-3.28 (m, 2H), 2.91-2.75 (m, 3H), 2.39 (t, J=6.9 Hz, 2H), 2.30 (br d, J=12.9 Hz, 1H), 2.14 (dd, J=12.3, 2.4 Hz, 1H), 1.89-1.67 (m, 6H), 1.58-1.40 (m, 3H), 1.26 (s, 3H), 1.217 (s, 3H), 1.216 (d, J=6.9 Hz, 6H). ¹³CNMR (CDCl₃, 75 MHz) δ 179.4, 177.7, 147.1, 145.9, 134.7, 127.0, 124.2, 124.0, 47.5, 45.5, 39.4, 38.1, 37.4, 37.2, 33.6, 31.8, 30.1, 25.3, 24.9, 24.1, 21.3, 18.9, 16.6. MS (ESI⁻): m/z calcd for C₂₄H₃₄NO₃ (M-H⁻) 384.25, found 384.34.

Wu151: ¹HNMR (CDCl₃, 300 MHz) δ 7.15 (d, J=8.1 Hz, 1H), 6.98 (dd, J=8.1, 1.5 Hz, 1H), 6.86 (d, J=1.5 Hz, 1H), 6.37 (t, J=5.1 Hz, 1H), 3.39-3.18 (m, 4H), 2.90-2.74 (m, 3H), 2.44-2.03 (m, 6H), 1.90-1.66 (m, 8H), 1.59-1.38 (m, 3H), 1.26 (s, 3H), 1.210 (s, 3H), 1.209 (d, J=6.9 Hz, 6H). ¹³CNMR (CDCl₃, 75 MHz) δ 179.9, 173.5, 147.1, 145.9, 134.7, 127.0, 124.2, 124.1, 47.5, 45.5, 39.5, 39.4, 38.1, 37.4, 37.2, 33.9, 33.6, 30.1, 25.8, 25.3, 24.1, 21.3, 18.8, 16.7. MS (ESI⁻): m/z calcd for C₂₈H₄₁N₂O₄ (M-H⁻) 469.31, found 469.40.

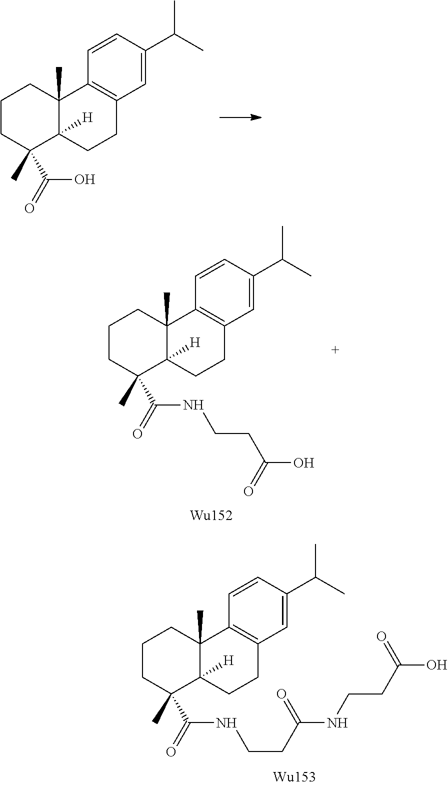

### 3-((1R,4aS,10aR)-7-isopropyl-1,4a-dimethyl-1,2,3,4,4a,9,10,10a-octahydrophenanthrene-1-carboxamido)propanoic Acid (Wu152) and 3-(3-((1R,4aS,10aR)-7-isopropyl-1,4a-dimethyl-1,2,3,4,4a,9,10,10a-octahydrophenanthrene-1-carboxamido)propanamido)propanoic Acid (Wu153)

Followed the general procedure of amide coupling, the reaction mixture was purified using preparative LC (B/A: 10:90 to 80:20) to give Wu153 (7.2 mg, 7%) and Wu152 (29.6 mg, 34%). Wu152: ¹HNMR (CDCl₃, 300 MHz) δ 7.15 (d, J=8.1 Hz, 1H), 6.99 (dd, J=8.1, 1.8 Hz, 1H), 6.86 (d, J=1.8 Hz, 1H), 6.50 (br s, 1H), 3.61-3.41 (m, 2H), 2.90-2.73 (m, 3H), 2.65-2.49 (m, 2H), 2.30 (br d, J=12.6 Hz, 1H), 2.12 (d, J=10.8 Hz, 1H), 1.84-1.65 (m, 4H), 1.58-1.38 (m, 3H), 1.25 (s, 3H), 1.212 (s, 3H), 1.215 (d, J=6.9 Hz, 6H). ¹³CNMR (CDCl₃, 75 MHz) δ 179.2, 176.9, 147.0, 145.9, 134.7, 127.0, 124.2, 124.0, 47.4, 45.5, 38.1, 37.3, 37.2, 35.4, 34.0, 33.6, 30.1, 25.4, 24.1, 21.3, 18.8, 16.6. MS (ESI⁻): m/z calcd for C₂₃H₃₂NO₃ (M-H⁻) 370.24, found 370.35.

Wu153: ¹HNMR (CDCl₃, 300 MHz) δ 7.14 (d, J=8.4 Hz, 1H), 7.04 (t, J=5.4 Hz, 1H), 6.98 (dd, J=8.4, 1.8 Hz, 1H), 6.85 (d, J=1.8 Hz, 1H), 6.79 (t, J=5.7 Hz, 1H), 3.56-3.39 (m, 4H), 2.88-2.72 (m, 3H), 2.59-2.33 (m, 4H), 2.28 (d, J=12.6 Hz, 1H), 2.08 (dd, J=12.6, 2.1 Hz, 1H), 1.83-1.64 (m, 4H), 1.56-1.35 (m, 3H), 1.24 (s, 3H), 1.21 (d, J=6.9 Hz, 6H) 1.20 (s, 3H). ¹³CNMR (CDCl₃, 75 MHz) δ 180.1, 175.2, 172.1, 147.0, 145.9, 134.7, 127.0, 124.2, 124.1, 47.5, 45.4, 38.1, 37.3, 37.2, 36.8, 35.6, 34.9, 33.9, 33.6, 30.0, 25.4, 24.1, 21.3, 18.8, 16.6. MS (ESI⁻): m/z calcd for C₂₆H₃₇N₂O₄ (M-H⁻) 441.28, found 441.37.

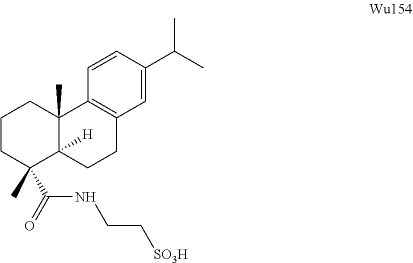

### 2-((1R,4aS,10aR)-7-isopropyl-1,4a-dimethyl-1,2,3,4,4a,9,10,10a-octahydrophenanthrene-1-carboxamido)ethane-1-sulfonic Acid (Wu154)

Followed the general procedure of amide coupling, the reaction mixture was purified using preparative LC (B/A: 15:85 to 100:0) to give fractions with right mass ion. Because neutral condition was used for the purification and the product was quite acidic. To the concentrated fractions was added 2 mL water, acidified with 1N HCl to pH˜1, extracted with ethyl acetate (4 mL×4), concentrated to give Wu154 (32.1 mg, 34%). ¹HNMR (CDCl₃, 300 MHz) δ 9.02 (br s, 1H), 8.23 (br s, 1H), 7.01 (d, J=8.4 Hz, 1H), 6.94 (dd, J=8.4, 1.2 Hz, 1H), 6.81 (d, J=1.2 Hz, 1H), 3.87-3.59 (m, 2H), 3.25-3.03 (m, 2H), 2.86-2.69 (m, 3H), 2.23-1.99 (m, 2H), 1.80-1.44 (m, 5H), 1.41-1.23 (m, 2H, not including methyl group), 1.26 (s, 3H), 1.19 (d, J=6.9 Hz, 6H), 1.12 (s, 3H). ¹³CNMR (CDCl₃, 75 MHz) δ 181.7, 146.7, 145.8, 134.4, 126.9, 124.2, 124.0, 49.7, 47.6, 44.7, 37.6, 37.1, 36.9, 36.4, 33.6, 29.9, 25.2, 24.1, 21.2, 18.5, 16.3. MS (ESI⁻): m/z calcd for C₂₂H₃₂NO₄S (M-H⁻) 406.21, found 406.33.

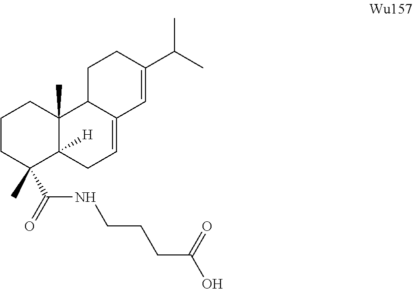

### 4-((1R,4aR,4bR,10aR)-7-isopropyl-1,4a-dimethyl-1,2,3,4,4a,4b,10,10a-decahydrophenanthrene-1-carboxamido)butanoic Acid (Wu157)

Followed the synthesis and purification procedure for Wu149, instead of dehydroabietic acid, abietic acid (70 mg, 0.231 mmol) with 70% technical purity was used and compound Wu157 (15 mg, 17%) was obtained. ¹HNMR (CDCl₃, 300 MHz) δ 6.09 (t, J=5.7 Hz, 1H), 5.74 (s, 1H), 5.35-5.29 (m, 1H), 3.40-3.18 (m, 2H), 2.37 (t, J=6.9 Hz, 2H), 2.21 (m, 1H), 2.10-1.69 (m, 11H), 1.62-1.47 (m, 3H), 1.25 (s, 3H), 1.24-1.09 (m, 2H), 1.10 (d, J=6.9 Hz, 3H), 0.99 (d, J=6.9 Hz, 3H), 0.82 (s, 3H). ¹³C-NMR (75 MHz, CDCl₃) δ 179.8, 168.1, 145.4, 135.7, 122.6, 120.6, 51.1, 46.5, 45.7, 39.4, 38.4, 37.7, 35.0, 34.8, 31.8, 27.6, 25.5, 25.0, 22.6, 21.6, 21.0, 18.4, 17.1, 14.3. MS (ESL): m/z calcd for C24H₃₆NO₃ (M-H⁻) 386.27, found 386.48.

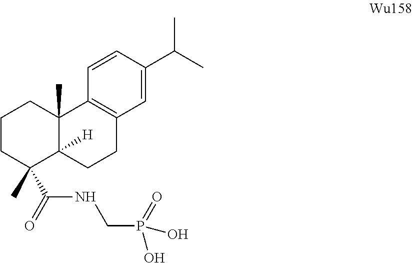

### (((1R,4aS,10aR)-7-isopropyl-1,4a-dimethyl-1,2,3,4,4a,9,10,10a-octahydrophenanthrene-1-carboxamido)methyl)phosphonic Acid (Wu158)

Followed the general procedure of amide coupling, 10 equiv of DiPEA was used. The reaction mixture was stirred at rt overnight, little amount product was found. The reaction mixture was then stirred at 50° C. for another night. Following the purification procedure for Wu154. After concentration, to the product was added 2 mL water, acidified with 1N HCl to pH ˜2, extracted with ethyl acetate (4 mL×4), concentrated to give Wu158 (8.7 mg, 9%). ¹HNMR (CDCl₃, 300 MHz) δ 8.23 (br s, 2H), 7.14 (br s, 1H), 7.11 (d, J=8.4 Hz, 1H), 6.95 (dd, J=8.4, 1.5 Hz, 1H), 6.83 (d, J=1.5 Hz, 1H), 3.85-3.50 (m, 2H), 2.90-2.67 (m, 3H), 2.24 (br d, J=12.6 Hz, 1H), 2.13 (d, J=12.0 Hz, 1H), 1.82-1.34 (m, 7H), 1.26 (s, 3H), 1.18 (d, J=6.9 Hz, 6H), 1.15 (s, 3H). ¹³CNMR (CDCl₃, 75 MHz) δ 181.7, 146.9, 145.8, 134.8, 127.1, 124.1, 124.0, 47.7, 45.4, 38.0, 37.1, 36.9, 33.6, 30.1, 25.4, 24.15, 24.09, 21.2, 18.7, 16.5. ³¹PNMR (CDCl₃, 121 MHz) δ 23.24. MS (ESI⁻): m/z calcd for C21H₃₁NO₄P (M-H⁻) 392.20, found 392.28.

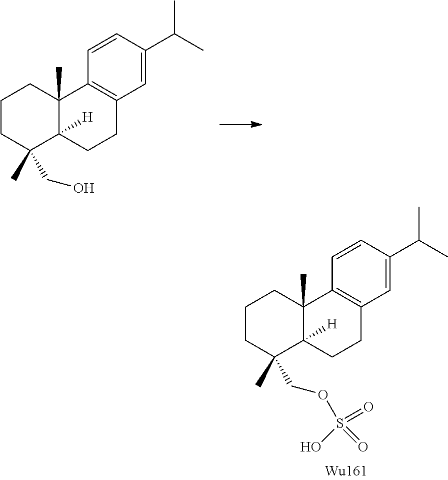

### ((1R,4aS,10aR)-7-isopropyl-1,4a-dimethyl-1,2,3,4,4a,9,10,10a-octahydrophenanthren-1-yl)methyl Hydrogen Sulfate (Wu161)

The reaction mixture of alcohol (35 mg, 0.122 mmol) and PySO₃ (36.7 mg, 0.232 mmol) in THF/DMF (2 mL/1 mL) was stirred at rt for about 2 h, concentrated and purified on preparative LC, extracted with EtOAc after adjusting with HCl to pH 4-5. Concentrated to give Wu161 (22.5 mg, 50%). ¹HNMR (CDCl₃, 300 MHz) δ 8.32 (br s, 1H), 7.14 (d, J=8.2 Hz, 1H), 6.96 (dd, J=8.2, 1.9 Hz, 1H), 6.86 (d, J=1.9 Hz, 1H), 3.92 (d, J=9.0 Hz, 1H), 3.73 (d, J=9.0 Hz, 1H), 2.90-2.75 (m, 3H), 2.25 (br d, J=12.8 Hz, 1H), 1.80-1.60 (m, 5H), 1.50-1.32 (m, 3H), 1.21 (d, J=6.9 Hz, 6H), 1.19 (s, 3H), 0.93 (s, 3H). ¹³CNMR (CDCl₃, 75 MHz) δ 147.1, 145.7, 134.9, 126.9, 124.3, 123.9, 78.2, 44.1, 38.4, 37.5, 37.2, 35.2, 33.6, 30.1, 25.4, 24.1, 19.1, 18.6, 17.2. MS (ESI⁻): m/z calcd for C₂₀H₂₉O₄S (M-H⁻) 365.18, found 365.25.

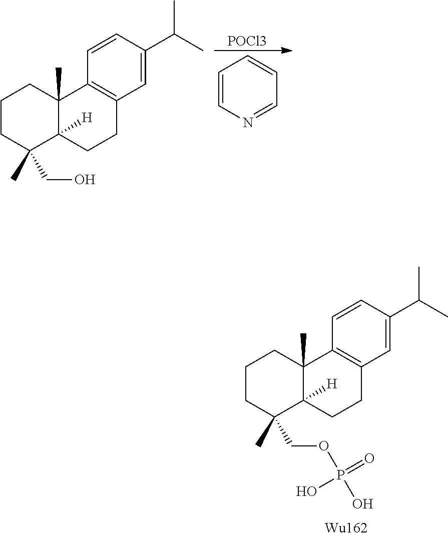

### ((1R,4aS,10aR)-7-isopropyl-1,4a-dimethyl-1,2,3,4,4a,9,10,10a-octahydrophenanthren-1-yl)methyl Dihydrogen Phosphate (Wu162)

To the alcohol (41 mg, 0.143 mmol) in THF (2 mL) added pyridine (90.5 mg, 1.14 mmol), followed by POCl₃ (24.1 mg, 0.157 mmol) at 0° C. The mixture was stirred at rt for 2 h. Then 1 mL 2N HCl aqueous solution was added and stirred at rt overnight. Desired compound was found according to LC, 2 mL water was added to the mixture and extracted with EtOAc 5 mL×3. Concentrated and purified with preparative LC (B/A: 15:85 to 100:0). The desired fractions were combined and acidified with 1N HCl (1.2 mL) to about pH 2, 5 mL water was added and extracted with EtOAc (5 mL×4), concentrated to give Wu162 (38.7 mg, 74%). ¹HNMR (CDCl₃, 300 MHz) δ 9.19 (br s, 2H), 7.17 (d, J=8.1 Hz, 1H), 6.99 (dd, J=8.1, 1.8 Hz, 1H), 6.88 (d, J=1.8 Hz, 1H), 3.91-3.80 (m, 1H), 3.70-3.60 (m, 1H), 2.90-2.75 (m, 3H), 2.15 (d, J=12.9 Hz, 1H), 1.80-1.35 (m, 8H), 1.23 (d, J=6.3 Hz, 6H), 1.21 (s, 3H), 0.92 (s, 3H). ¹³CNMR (CDCl₃, 75 MHz) δ 147.2, 145.7, 134.9, 127.0, 124.4, 124.0, 76.1 (br d, J_{CP}=5.0 Hz), 43.7, 38.3, 37.5, 37.45 (br d, J_{CP}=7.5 Hz), 35.0, 33.6, 30.2, 25.5, 24.2, 24.1, 19.0, 18.6, 17.1. ³¹PNMR (CDCl₃, 121 MHz) δ 2.14. MS (ESI⁻): m/z calcd for C₂₀H₃₀O₄P (M-H⁻) 365.19, found 365.25.

Synthesis of Wu176 and Wu179

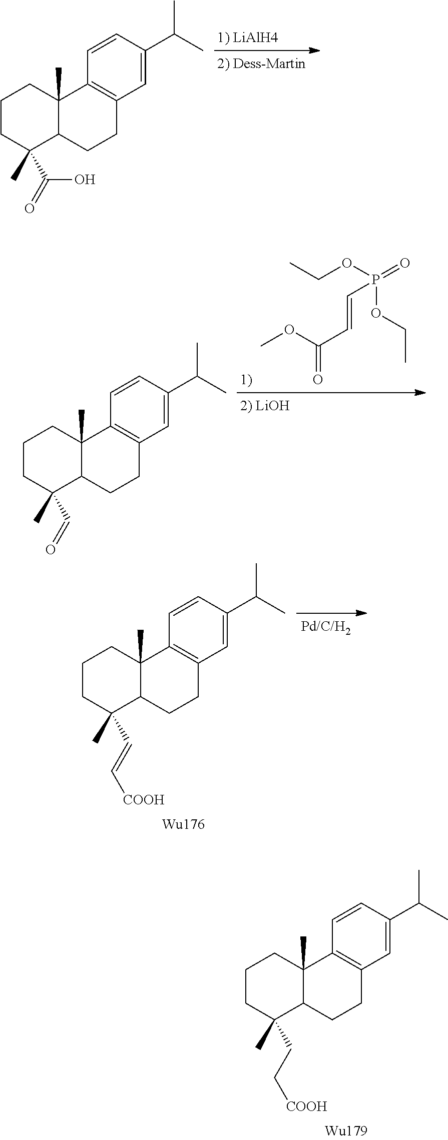 [SMILES: CC(C)c1ccc2c(c1)CCC1[C@](C)(C(=O)O)CCC[C@]21C ; CC(C)c1ccc2c(c1)CCC1[C@](C)(C=O)CCC[C@]21C ; CCOP(=O)(/C=C/C(=O)OC)OCC ; CC(C)c1ccc2c(c1)CCC1[C@](C)(/C=C/C(=O)O)CCC[C@]21C ; CC(C)c1ccc2c(c1)CCC1[C@](C)(CCC(=O)O)CCC[C@]21C]

Step 1: Synthesis of Dehydroabietyl Aldehyde

To dehydroabietic acid (140 mg, 0.466 mmol) in anhydrous THF (8 mL) added LiAlH₄ (1.5 mmol) and stirred at rt for about 40 min, the reaction was quenched with ice-water dropwise until no bubble was formed, filtered and washed with DCM/MO (3:1, 10 mL). Combined and concentrated, re-dissolve in DCM and concentrated to give a raw product, full conversion according to HNMR.

To the mixture of raw product (0.466 mmol) and Dess-Martin reagent (217.4, 0.513 mmol) added dry DCM (15 mL) and stirred at rt for about 2 h. Full conversed based on LC, pre-adsorbed on silica gel and purified using EtOAc/n-heptane (20:80) to give product (125 mg, 94%). ¹HNMR (CDCl₃, 300 MHz) δ 9.28 (s, 1H), 7.20 (d, J=8.1 Hz, 1H), 7.03 (dd, J=8.1, 1.8 Hz, 1H), 6.92 (d, J=1.8 Hz, 1H), 2.95-2.76 (m, 3H), 2.36 (dt, J=13.5, 3.3 Hz, 1H), 1.98-1.74 (m, 4H), 1.55-1.41 (m, 2H), 1.40-1.29 (m, 2H), 1.25 (br s, 3H), 1.24 (d, J=6.9 Hz, 6H), 1.18 (s, 3H). ¹³CNMR (CDCl₃, 75 MHz) δ 206.3, 146.3, 146.1, 134.5, 127.1, 124.3, 124.1, 49.9, 42.9, 38.0, 36.4, 33.6, 32.1, 29.9, 25.3, 24.11, 24.09, 21.5, 17.9, 14.1.

Step 2: Wittig Reaction Followed by Hydrolysis

### (E)-3-((1S,4aS)-7-isopropyl-1,4a-dimethyl-1,2,3,4,4a,9,10,10a-octahydrophenanthren-1-yl)acrylic Acid (Wu176)

To the mixture of methyldiethylphosphonic acetate (33.3 mg, 0.158 mmol) and NaH (3.8 mg, 0.158 mmol) added 0.7 mL THF and stirred at rt for 30 min. The solution of dehydroabietyl aldehyde (30 mg, 0.105 mmol)) in THF (750 uL) was added to the reaction mixture and stirred at rt for 3 h. HPLC indicated part of the acid (Wu176) was formed together with methyl ester as major. LiOH.H₂O (25 mg, 0.597 mmol) was then added, followed by 0.4 mL water and 0.8 mL THF. The mixture was irradiated under MW for 25 min at 100° C. Full conversion was achieved. Acidified using 1N HCl to pH˜5, extracted with EtOAc (3 mL×4), concentrated and purified using preparative LC (B/A:30:70 to 100:0) to give Wu176 (28 mg, 82%). ¹HNMR (CDCl₃, 300 MHz) δ 7.17 (d, J=8.4 Hz, 1H), 7.01 (dd, J=8.4, 1.8 Hz, 1H), 6.96 (d, J=15.9 Hz, 1H), 6.89 (d, J=1.8 Hz, 1H), 5.80 (d, J=15.9 Hz, 1H), 2.93-2.74 (m, 3H), 2.34 (br d, J=12.9 Hz, 1H), 1.87-1.34 (m, 8H), 1.25 (s, 3H), 1.22 (d, J=6.9 Hz, 6H), 1.17 (s, 3H). ¹³CNMR (CDCl₃, 75 MHz) δ 172.6, 164.0, 147.0, 145.9, 134.8, 127.1, 124.2, 124.1, 117.9, 47.7, 41.1, 38.8, 38.3, 37.1, 33.6, 30.1, 25.4, 24.1, 20.5, 18.7, 17.6. MS (ESI⁻): m/z calcd for C₂₂H₂₉O₂ (M-H⁻) 325.22, found 325.27.

Step 3: Hydrogenation

### 3-((1S,4aS)-7-isopropyl-1,4a-dimethyl-1,2,3,4,4a,9,10,10a-octahydrophenanthren-1-yl)propanoic Acid (Wu179)

The mixture of Wu176 (17 mg, 0.052 mmol) and 10% Pd/C (2.8 mg, 0.0026 mmol) in 3 mL THF was stirred at rt under atm H2 for 72 h. The conversion was not completed. The reaction mixture was filtered, concentrated and purified using preparative LC (B/A: 30:70 to 100:0) to give Wu179 (12.4 mg, 73%). ¹HNMR (CDCl₃, 300 MHz) δ 7.18 (d, J=8.1 Hz, 1H), 7.00 (dd, J=8.1, 1.8 Hz, 1H), 6.90 (d, J=1.8 Hz, 1H), 2.98-2.76 (m, 3H), 2.34-2.23 (m, 3H), 1.89-1.54 (m, 6H), 1.47-1.31 (m, 3H), 1.28-1.15 (m, 10H), 0.95 (s, 3H). ¹³CNMR (CDCl₃, 75 MHz) δ 180.7, 147.5, 145.7, 134.8, 127.0, 124.4, 124.0, 48.0, 38.6, 37.7, 37.0, 35.6, 33.6, 30.4, 28.8, 25.4, 24.1, 20.4, 19.0, 18.8. MS (ESI⁻): m/z calcd for C₂₂H₃₁O₂ (M-H⁻) 327.23, found 327.30.

Synthesis of Wu180

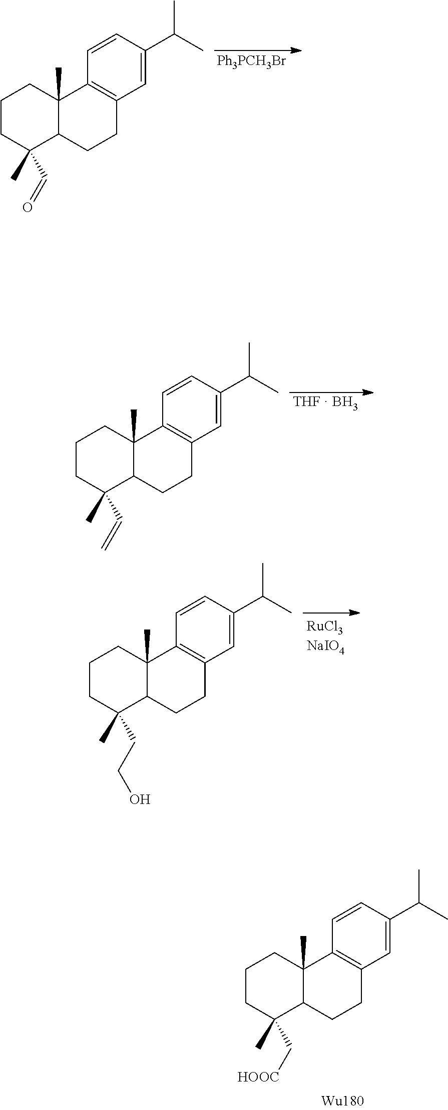

Step 1: Wittig Reaction

The mixture of Ph₃PCH₃Br (75.4 mg, 0.211 mmol) and NaH (5 mg, 0.211 mmol) in 0.7 mL THF was stirred at 50° C. for 15 min. The dehydroabietyl aldehyde (40 mg, 0.141 mmol) was added and stirred at 50° C. overnight. The reaction mixture was purified using preparative LC with eluent (B/A: 100:0, 10 mM NH4OAc) to give the alkene (40 mg, 100%). ¹HNMR (CDCl₃, 300 MHz) δ 7.18 (d, J=8.1 Hz, 1H), 7.00 (dd, J=8.1, 1.8 Hz, 1H), 6.89 (d, J=1.8 Hz, 1H), 5.65 (dd, J=17.1, 11.4 Hz, 1H), 5.00-4.92 (m, 2H), 2.93-2.72 (m, 3H), 2.35-2.27 (m, 1H), 1.88-1.57 (m, 4H), 1.51-1.30 (m, 4H), 1.24 (s, 3H), 1.23 (d, J=6.9 Hz, 6H), 1.08 (s, 3H). ¹³CNMR (CDCl₃, 75 MHz) δ 151.7, 147.6, 145.7, 135.1, 127.1, 124.3, 124.0, 111.0, 48.2, 40.4, 39.9, 38.7, 37.3, 33.6, 30.5, 25.5, 24.1, 19.9, 19.1, 17.3.

Step 2: Hydroboration

To the resulted alkene (40 mg, 0.142 mmol) from last step in 2 mL THF added 1 mL 1 M borane in THF. The solution was stirred at rt overnight. The conversion was not completed due to the poor quality of borane. The reaction was still quenched with 2 mL water, followed by 0.5 mL 1M NaOH and 1 mL 30% H₂O₂. The mixture was stirred overnight. The reaction mixture was extracted with DCM (3 mL×3), concentrated and purified using silica gel chromatography with EtOAc/n-heptane (10:90 to 25:75) to give an alcohol (13 mg, 30%). ¹HNMR (CDCl₃, 300 MHz) δ 7.17 (d, J=8.1 Hz, 1H), 7.00 (dd, J=8.1, 1.8 Hz, 1H), 6.90 (d, J=1.8 Hz, 1H), 3.69 (t, J=7.5 Hz, 2H), 2.97-2.74 (m, 3H), 2.27 (m, 1H), 1.92-1.56 (m, 6H), 1.47-1.32 (m, 4H), 1.23 (d, J=6.9 Hz, 6H), 1.21 (s, 3H), 0.97 (s, 3H). ¹³CNMR (CDCl₃, 75 MHz) δ 147.6, 145.7, 134.9, 127.0, 124.4, 124.0, 59.4, 48.3, 46.9, 38.6, 37.9, 37.7, 35.8, 33.6, 30.5, 25.5, 24.1, 21.0, 19.1, 19.0.

Step 3: Oxidation

### 2-((1 S,4aS)-7-isopropyl-1,4a-dimethyl-1,2,3,4,4a,9,10,10a-octahydrophenanthren-1-yl)acetic Acid (Wu180)

The mixture of alcohol (13 mg, 0.0433 mmol) from last step, RuCl₃ (0.9 mg, 0.00433 mmol) and NalO₄ (13.9 mg, 0.0650 mmol) in 1.5 mL solvent mixture CCl₄/CH₃CN/Water (0.5:0.5:0.5) was stirred at rt for 5 h. Quenched with 1 N HCl and ice. Extracted with DCM (3 mL×3), concentrated and purified using preparative LC (B/A: 30:70 to 100:0) to give Wu180 (6.2 mg, 46%). ¹HNMR (CDCl₃, 300 MHz) δ 7.16 (d, J=8.1 Hz, 1H), 6.99 (dd, J=8.1, 1.8 Hz, 1H), 6.89 (d, J=1.8 Hz, 1H), 2.95-2.76 (m, 3H), 2.32 (s, 2H), 2.27 (dt (J=12.6, 3.0 Hz, 1H), 1.90-1.49 (m, 7H), 1.45-1.33 (m, 1H), 1.25-1.20 (m, 9H), 1.07 (s, 3H). ¹³CNMR (CDCl₃, 75 MHz) δ 177.6, 147.3, 145.7, 134.9, 127.0, 124.4, 124.0, 48.3, 38.4, 38.0, 37.6, 37.5, 33.6, 30.4, 25.5, 24.1, 20.8, 19.5, 19.0. MS (ESI⁻): m/z calcd for C₂₁H₂₉O₂ (M-H⁻) 313.22, found 313.20.

### ((1R,4aS,10aR)-6,7,8-trichloro-1,4a-dimethyl-2,3,40,10,10a-hexahydrophenanthrene-1-yl)methyl Hydrogen Sulfate (Wu181)

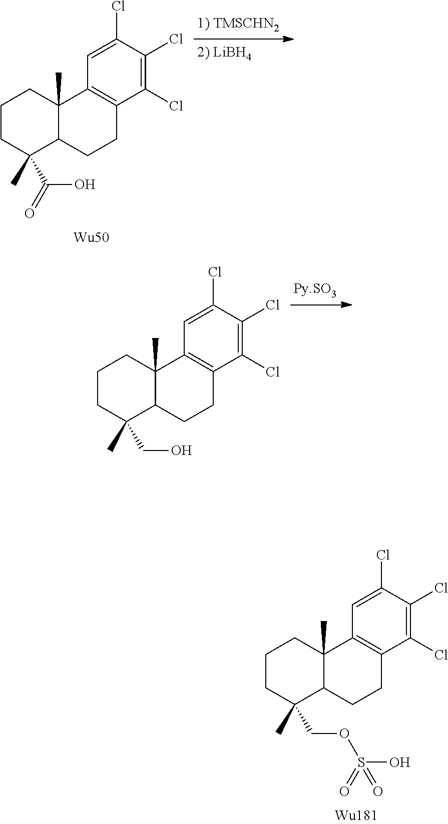 [SMILES: C[C@@]1(C(=O)O)CCC[C@]2(C)c3cc(Cl)c(Cl)c(Cl)c3CCC12 ; C[C@@]1(CO)CCC[C@]2(C)c3cc(Cl)c(Cl)c(Cl)c3CCC12 ; C[C@@]1(COS(=O)(=O)O)CCC[C@]2(C)c3cc(Cl)c(Cl)c(Cl)c3CCC12]

Wu50 was synthesized according to methods described in WO 2016/114707. To the substance Wu50 (23 mg, 0.0636 mmol) added MeOH/toluene (1/1.5 mL), followed by 2M Me₃SiCHN₂ (70 uL, 0.140 mmol) in hexane. Full conversion was achieved after 2 h. The reaction was then quenched with 2 drops of HOAc, concentrated and co-evaporated with 2 mL toluene. The crude product was dissolved in 1 mL anhydrous THF, and LiBH₄ (4.2 mg, 0.191 mmol) was then added. The mixture was stirred at rt overnight. The reaction was quenched with methanol and pre-adsorbed on silica gel, purified using EA-n-heptane (20:80 to 30:70) to give the alcohol (20.5 mg, 93%). ¹HNMR (CDCl₃, 300 MHz) δ 7.30 (s, 1H), 3.49 (d, J=10.8 Hz, 1H), 3.12 (d, J=10.8 Hz, 1H), 3.01-2.89 (m, 1H), 2.81-2.66 (m, 1H), 2.25-2.16 (m, 1H), 1.96-1.85 (m, 1H), 1.82-1.55 (m, 4H), 1.52-1.27 (m, 4H), 1.20 (d, J=0.9 Hz, 3H), 0.87 (d, J=0.6 Hz, 3H). ¹³CNMR (CDCl₃, 75 MHz) δ 151.0, 134.3, 133.8, 130.8, 128.4, 124.8, 71.8, 42.6, 38.6, 38.02, 37.95, 34.9, 29.4, 25.3, 18.6, 18.4, 17.6.

Step 2: Formation of Wu181

The reaction mixture of alcohol (10 mg, 0.0288 mmol) and PySO₃ salt (18.2 mg, 0.115 mmol) in THF/DMF (1.5/0.5 mL) was stirred at rt for 1 h. Full conversion was achieved according to LC analysis. The mixture was concentrated and purified using preparative LC (B/A: 10:90 to 70:30) to give Wu181 (10 mg, 81%). ¹HNMR (CDCl₃, 300 MHz) δ 7.262 (s, 1H), 3.91 (br d, J=9.0 Hz, 1H), 3.64 (br d, J=9.0 Hz, 1H), 2.91 (dd, J=18.3, 6.0 Hz, 1H), 2.76-2.58 (m, 1H), 2.18-2.06 (m, 1H), 1.96-1.82 (m, 1H), 1.74-1.18 (m, 7H), 1.14 (s, 3H), 0.87 (s, 3H). ¹³CNMR (CDCl₃, 75 MHz) δ 150.7, 134.3, 133.8, 130.8, 128.4, 124.8, 77.0, 42.6, 38.3, 38.0, 37.1, 34.8, 29.2, 25.2, 18.4, 17.3. MS (ESL): m/z calcd for C₁₇H₂₀³⁵Cl₃O₄S (M-H⁻) 425.01, found 425.18; m/z calcd for C₁₇H₂₀³⁵Cl₂³⁷ClO₄S (M-H⁻) 427.01, found 427.10; m/z calcd for C₁₇H₂₀³⁵Cl³⁷Cl₂O₄S (M-H⁻) 429.01, found 429.01.

### FIGURE LEGENDS

FIG. 1. Lipoelectric compounds. (A) A compound binds with its hydrophobic anchor in the lipid membrane. The effector (a charged group) electrostatically affects the positively charged voltage sensor (S4). (B) Compounds in A affects the voltage-dependence of the channel opening. The direction of the shift depends on the valence of the charge. (C) Structure and nomenclature of dehydroabietic acid (DHAA). (D) Functional pH dependence for the effect of DHAA on the 3R Shaker Kv channel, from Ottosson, N. E. et al. Sci. Rep. 5, 13278 (2015). The carboxyl group in either protonated or deprotonated form.

FIG. 2. Effect of DHAA derivatives with different stalk length on the 3R Shaker Kv channel. (A) The Shaker Kv channel, top view of the VSD. Gating charges (arginines) as sticks: the wt Shaker Kv channel has 362R as top charge (=R1); the 3R Shaker Kv channel has two additional charges (M356R and A359R). (B) Nomenclature for the position of the charged group (effector) (P1-11) on the stalk (C) K currents at 10 mV and normalized G(V) curves, before (black) and after (grey) application of 100 μM P1-DHAA (shift: −13.0 mV) and P6-Wu149 (shift: −5.3 mV). pH=7.4. (D) G(V) shifting effect of DHAA derivatives with different stalk lengths and carboxyl group as effector. 100 μM, pH=7.4. Mean±SEM (n=3-11). Data fitted with a one phase exponential decay curve. Length constant=5.1 atoms.

FIG. 3. Role of stalk length with a fully charged effector. All compounds 100 μM. (A) Structures of permanently charged DHAA derivatives with a sulfonic-acid group. (B) G(V) shifting effects of DHAA derivatives with carboxyl groups (from FIG. 2B) at pH=10 (white symbols), and permanently charged groups (from a) at pH=7.4 (black symbols). Mean±SEM (n=3-7). Grey dashed line is for DHAA derivatives with carboxyl group at pH=7.4 (adopted from FIG. 2D). (C) Functional pH dependence for G(V) shifts of indicated compounds on the 3R Shaker Kv channel. Mean±SEM (n=3-11). pKₐ=7.2 (DHAA), 7.3 (Wu180), 7.5 (Wu179). 6.8 (Wu176). (D) Functional pH dependence for the effect of Wu154 on the 3R Shaker Kv channel. Mean±SEM (n=4-6). pKa<7.4. (E) G(V) shifting effects on the wt Shaker Kv channel. Symbols as in B. Mean±SEM (n=3-7).

FIG. 4. The cut-off model. (A) Schematic illustration of cut-off model. (B) Electrostatic energy for a charge q₁=1e at (0,4) nergy q2=−1e at (x,z) z. (C) Critical stalk length for a stalk fixed at (x,z) z) cal staq₁=1e at d=(0,2), (0,4), (0,6) for aq₂=−1e at the end of the stalk with different lengths. (D) Positions for the cut-off point when the stalk length is 4 h/=4 ions for thq₁=1e at d=(0,2), (0,4), (0,6) e cut-q₂=−1e at the end of the stalk.

FIG. 5. The valance of the charge is critical for the effect The 3R Shaker Kv channel. Concentration of compounds=100 the effect is 4). a carboxyl group. Ch(A) A)annel. Concentrate uncharged compounds. (B) Normalized G(V) curves. G(V) shift=0.0 mV. (C) G(V) shifts for DHAA derivatives in A. Mean 0 the efn=3-4). (D) Stalk structures of permanently charged compounds. (E) Normalized G(V) curves. G(V) shift=−34.6 mV. (F) G(V) shifts for DHAA derivatives in D. Mean ives (n=5-6).

FIG. 6. A divalent charge does not increase the G(V) shifting effect (A) Cut-off area for charge-dependent effects for a stalk length of 4 4 al=4 area for chq₁=1e at d=(0,4). q₂=−1e (black) and q₂=−2e (grey, dashed line). At point X, q₂=−1e will be attracted towards q₁=1e (representing the voltage sensor S4) in the membrane, and q₂=−2e will be attracted towards the water (with reduced effect on S4). (B) Structure of Wu162 and stalk structure for molecular species with valance 0, −1, −2. (C) Theoretical pH dependence for Wu162 (D) Functional pH dependence for Wu162 with valence −1 or −2. Mean ence (n=3-5). Dashed line: best fit of Eq. 5.

FIG. 7. Two carboxyl groups on the stalk. 3R shaker Kv channel, 100 μM (A) Structure of Wu148. (B) G(V) shifts for Wu148. pH=7.4. Compared to P1-DHAA, P4-Wu117, P5-Wu152. Mean±SEM (n=6), dashed lines. (C) Functional pH dependence for Wu148. Mean±SEM (n=3-6). G(V) shifts are compared with Wu148 at pH 7.4, one-way ANOVA Dunnett's multiple comparison test, *p<0.05.

FIG. 8. P3-Wu161 is five times more potent than DHAA. (A) Normalized G(V)-curves. G(V) shift=−10.4 mV. The 3R Shaker Kv channel. pH=7.4. (B) Concentration-response curves for P3-Wu161. pH=7.4. Mean±SEM (n=3-6). c_{1/2}=44 μM, ΔV_{MAX}=−13.9 mV (wt). c_{1/2}=36 μM, ΔV_{MAX}=−45.5 mV (3R). (C) Wu161 and DHAA on the 3R (10 μM) and wt (100 μM) Shaker Kv channels respectively. pH=7.4. Mean±SEM (n=3-10). Data for DHAA, from Ottosson, N. E. et al. Sci. Rep. 5, 13278 (2015).

FIG. 9. The role of S4 charges for the effect of Wu161 and other DHAA derivatives. Concentration for all compounds=100 μM. pH=7.4. (A) Schematic picture of S4 charges on the Shaker Kv channel. The top gating charge (an arginine, R362 in wt) was moved step-by-step further out on S4 (left), or removed (R362Q; right). (B) Wu161-induced G(V) shifts on the Shaker Kv channel S4 arginine mutants. Mean±SEM (n=3-5). Shifts are compared with R362Q (dashed line), one-way ANOVA with Dunnett.s multiple comparison test. Grey: non-significant, p>0.05. White: larger effect than for R362Q, p<0.001. Black: smaller effect than for R362Q, p<0.001. (C) Correlation between G(V) shifts for the PUFA DHA (Data for DHA: from Ottosson, N. E. et al. J. Gen. Physiol. 143, 173-182 (2014)) and for Wu161 on the Shaker Kv channel S4 arginine mutants. Slope (2.1±0.2) is significantly different from zero (Pearson correlation test and linear regression are both significant). (D) Effects of P1-DHAA, P1-Wu32 (Wu32 is described in WO 2016/114707), P3-Wu161 and PUFA DHA on the R362Q, wt and 3R Shaker Kv channels.

FIG. 10. Combined stalk and anchor modifications of the DHAA molecule. Effects on the 3R Shaker Kv channel. pH=7.4 (A) DHAA derivatives studied. (B) Concentration-response curves for DHAA derivatives in A. Mean±SEM (n=3-9). c_{1/2}=98 μM, ΔV_{MAX}=−26.5 mV (DHAA). c_{1/2}=36 μM, ΔV_{MAX}=−45.5 mV (Wu161). c_{1/2}=37 μM, ΔV_{MAX}=−46.0 mV (Wu50). c_{1/2}=6.1 μM, ΔV_{MAX}=−53.6 mV (Wu181). Data for Wu50 and DHAA, from Ottosson, N. E. et al. Sci. Rep. 5, 13278 (2015) (C) Currents at 10 mV and normalized G(V) curves. G(V) shift=−20.4 mV.

FIG. 11. DHAA derivatives open the human M-channel. (A) Currents before and after application of 100 μM Wu161. Steps to voltages between −120 and +20 mV start at t=0.7 s. At t=2.7 the voltage is switched to −10 mV. Traces at voltage=−20 mV in red. (B) G(V) curve for the cell in A. G(V) shift=−23.8 mV. (C) G(V) shifts for compounds with different stalk lengths (100 μM). Mean±SEM (n=3-6). P1-DHAA (pH=10). Permanently charged P2-Wu164, P3-Wu161, P5-Wu154 (pH=7.4). (D) Compounds with different valance (100 μM). Mean±SEM (n=3-5). ***p<0.001, significantly different from 0. (E) Concentration-response curves. Mean±SEM (n=3-4). c_{1/2}=41.8 μM, ΔV_{MAX}=−33.7 mV (Wu161). c_{1/2}=12.4 μM, ΔV_{MAX}=−42.1 mV (Wu181)

FIG. 12. Summary of suggested mechanisms. Proposed binding of four different compounds to the S3/S4 cleft. The three positive charges represent M356R, A359R, and R362R (=R1) in the top of S4. The negative charge represent the charged group of the four different compounds.

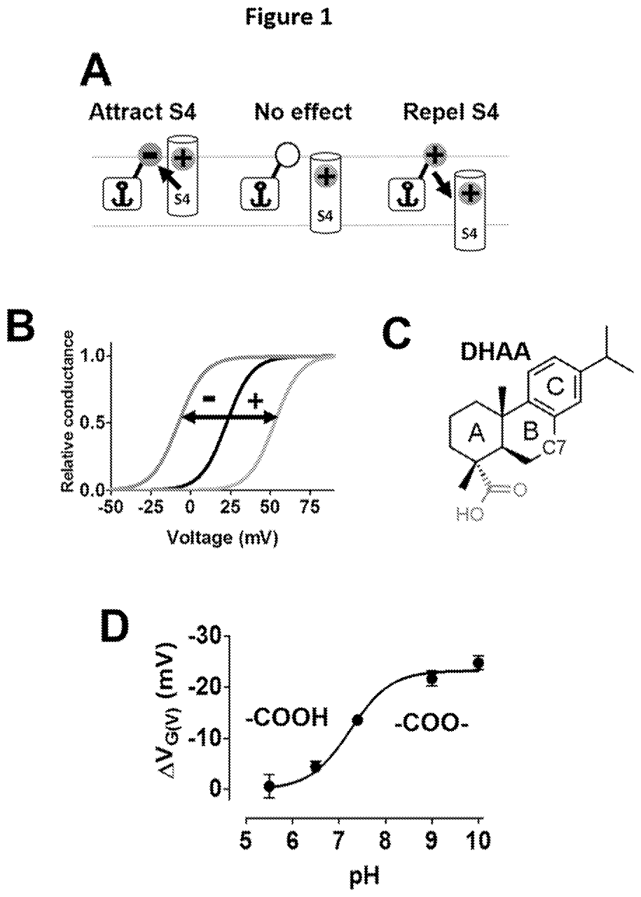

It is established that polyunsaturated fatty acids (PUFAs) bind to at least five different sites in different voltage-gated ion channels, see Elinder F & Liin SI Front. Physiol. 8:43 2017. One site found on the Drosophila Shaker Kv (Kv1-type) channel, and on the human Kv7.1 and Kv7.2/7.3 channels is close to S4. PUFAs anchored to this site, electrostatically open the channel via its negatively charged carboxyl group. The hydrophobic tail seems to act as an anchor for binding, and the charged group as the executor part, altering the S4 movement, the direction depending on the valence of the charge (FIG. 1A). A negatively charged group shifts the conductance-versus-voltage curve, G(V), in negative direction along the voltage axis, and a positively charged group in positive direction (FIG. 1B).

Hydrophobic resin acids (e.g. dehydroabietic acid (DHAA), FIG. 1C, and abietic acid (AA)), with a three-ring motif and a negatively charged carboxyl group most likely act via the same mechanism. The present inventors assume that the tree-ring motif acts as the anchor, and that the carboxyl group act as the executor. There are two major arguments for an electrostatic effect: (1) Altering the charge of the resin acid, or (2) altering the charge of the voltage sensor both affect the resin acid-induced G(V) shift. These two arguments can be divided in several subarguments: (1a) Altering pH protonated or deprotonated the carboxyl group and thereby altered the G(V) shift; the pKa value was 7.2 for DHAA (FIG. 1D). (1b) Altering the negatively charged carboxyl group to a positively charged amine group altered the sign of the G(V)shift. (2a) Addition of two positively charged arginines in the top of S4 of the Shaker Kv channel (M356R and A359R) increased the resin-acid induced G(V) shift about three-fold (This channel is referred to as the 3R Shaker Kv channel because the added charges together with the top charge in the wild-type (wt) Shaker Kv channel (R362) form the active triad of charges affecting the PUFA-induced shifts. (2b) The resin-acid charge supports or prevents rotation of the voltage sensor S4, depending on which side of S4 the top charge of S4 is located, and on the sign of the charge.

The inventors have previously performed a systematic exploration of molecular properties of the anchor of resin acids, primarily DHAA derivatives, when side chains on C7 in ring B (FIG. 1C) and halogenation of ring C was altered, see WO 2016/114707. The anchor modifications likely alter properties such as the depth of binding (into the lipid membrane), the affinity, the pKa value, and the solubility, which in turn affect the channel-opening properties. In the present invention, the effect of DHAA derivatives with modifications at the effector-site (carboxyl acid-site) is explored in an effort to maximize the interaction between the resin acid and the voltage sensor. In particular, the distance between the carboxyl group and S4 is considered (by putting a stalk between the carboxyl-group and the anchor with increasing length) and by increasing the valence of charge.

Increasing the Stalk Length Decreases the Effect on the 3R Channel at Neutral pH

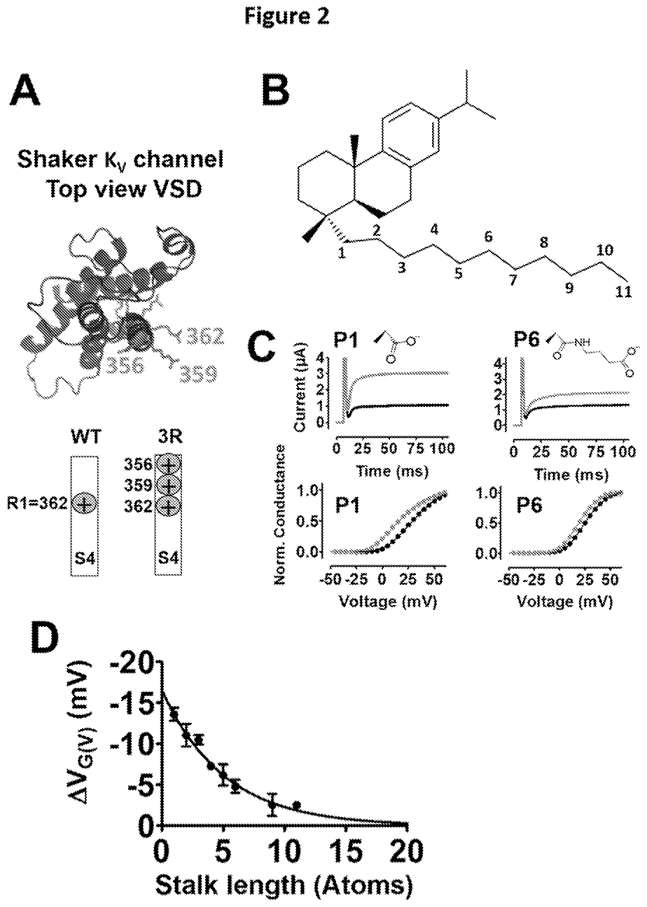

We hypothesized that a shorter distance between this negatively charged effector (carboxyl group) and the positively charged gating charges (arginines) of the voltage sensor S4 should increase the G(V)-shifting efficacy of the resin acid on the Shaker Kv channel. To explore this, we used the 3R Shaker Kv channel (with two additional arginines in the top of S4, M356R and A359R, FIG. 2A), a modification clearly increasing the resin-acid induced G(V) shift. Synthesized DHAA molecules with the carboxyl group located further and further out from the three-ringed motif (the anchor) were tested at pH 7.4 and 100 μM (FIG. 2B-D). When the stalk length increased, the resin-acid induced G(V) shift decreased exponentially (FIG. 2D). The length constant was 5.1±0.5 atoms. However, the functional pKa value (around 7.2 for DHAA) is sensitive to the local environment, and the local environment might be very different depending on the stalk length, e.g. further into the membrane or closer to extracellular solution. We therefore aimed to minimize the environmental influence (and the theoretical pKa differences) by making the effector fully charged.

### EXAMPLE 2

A Critical Cut-Off Point for Stalks Between Three and Four Atoms

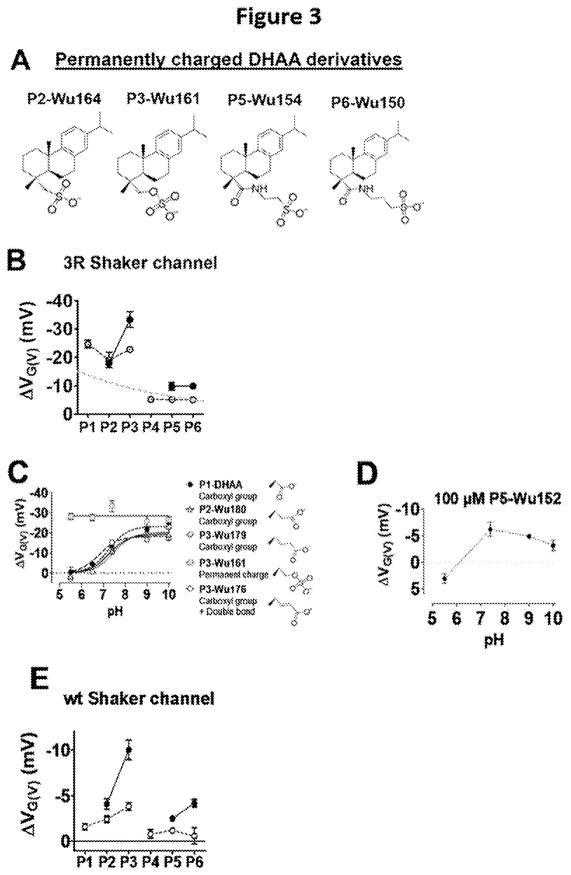

To explore the effect with a fully charged effector experiments were performed either at pH 10 to make sure that the carboxyl group, regardless of stalk length, was fully charged (see FIG. 1D), or alternatively, changed the carboxyl group to a permanently negatively charged sulfonic-acid group whenever possible (FIG. 3A). For stalk lengths up to three atoms (P1-P3) the effect was clearly potentiated at pH 10 (white symbols, FIG. 3B), or when a permanently charged group was used (black symbols, FIG. 3B), compared with pH 7.4 for the compounds with carboxyl groups (grey dashed curve, FIG. 3B). For the permanently charged compounds the effect was much larger for P3 than for P2. However, for stalks longer than three carbons the effect was radically suppressed (FIG. 3B) and the compounds with a carboxyl group were no longer sensitive to an increased pH. The carboxyl acid-derivatives with one, two- or three-carbon stalks had a functional pKₐ value of 7.2 (P1), 7.3 (P2), and 7.5 (P3), respectively, while the permanently charged P3 compound was not pH sensitive, as expected because of its low theoretical pKₐ value (FIG. 3C). By altering the stalk geometry it was also possible to shift the pKa value. Wu176 with a double bond in the stalk had a pKa value of 6.8 (FIG. 3C). The P4-P6 compounds had functional pKa values <7.4 as if these carboxyl groups experienced another local environment (FIG. 3D).

The data on the 3R Shaker Kv channel shows that (i) the largest effect was found for the permanently charged P3 DHAA derivative, and that (ii) there was a drastic decrease in effect beyond three carbons. A critical question is if this behaviour is found also for the wt Shaker Kv channel (with clear sequence similarity in S4 to several human Kv channels) with apparent therapeutic implications, or if it is restricted to the artificial 3R Shaker Kv channel. When the stalk-length series was tested on the wt Shaker Kv channel a noticeable difference, except for the absolute magnitude, was that derivative-induced G(V) shift increased when the stalk length was prolonged from P1 to P3, but the cut-off was not shifted (FIG. 3E). This suggests that the stalk length is a powerful variable to alter the efficacy for a channel-opening pharmaceutical drug. It also opens for a selectivity if, let us say, another channel, which is not intended to be affected by the drug, has a cut-off between P2 and P3.

If a longer stalk brings the negative charge closer to S4 and thereby increases channel opening and the G(V) shift (FIG. 3B) was corroborated for stalk lengths up to three atoms, most clearly for the wt Shaker Kv channel and in particular for permanently charged compounds at pH 7.4. However, the radically sharp cut-off at longer stalk lengths was a surprise.

### EXAMPLE 3

The Cut-Off Model

The experimental data presented above showed that the G(V)-shifting effect increased if the stalk was prolonged, as if the negative resin-acid charge was allowed to come closer to the positive S4 charge to more strongly pull the channel open. At a certain length the G(V)-shifting effect disappeared and a sudden break occurred in the curve (FIG. 3B,E). A simple explanation for this behaviour is that when the stalk exceeds a certain length the negative resin-acid charge suddenly find an energetically more suitable position, which is far away from the S4 charge, or even outside the membrane, and thus do not help to open the channel.

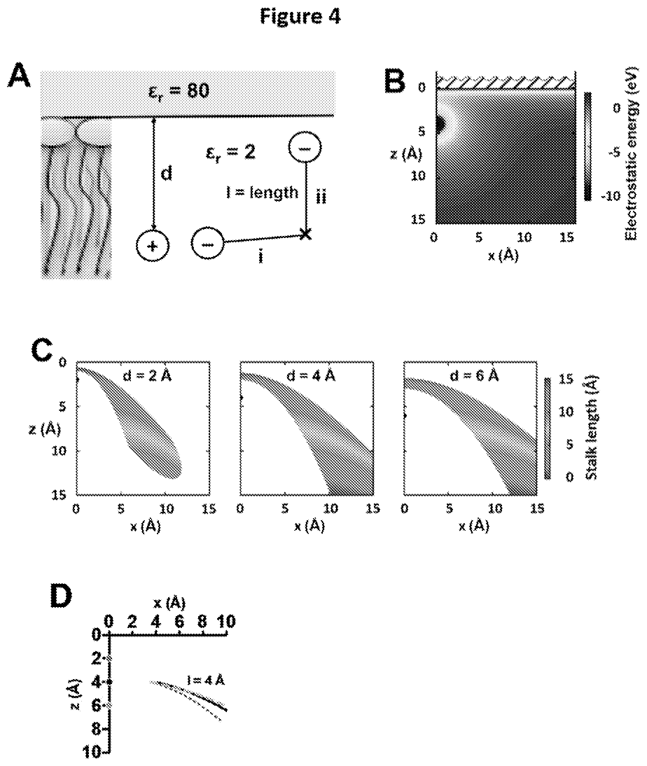

To explore this theoretically and quantitatively we analysed a simple system (FIG. 4A) where a low dielectric medium (the lipid bilayer, relative dielectric constant εᵣ=2) meets a high dielectric medium (water, εᵣ=80). In reality, the dielectric medium varies gradually in the channel's rough structure, but the model can give us some simple guidelines. For simplicity also there are no ions in the solution. A fixed positive charge in the lipid, at a distance d from the water represents the voltage sensor S4, most likely the top charge of S4. If a counter charge (of valence −1) is introduced at specific positions of the system, the total electrostatic energy can be calculated (d=4 Å in FIG. 4B). Next, if the counter charge instead is attached to a stalk and the other end of the stalk is fixed at a certain anchor point (x in FIG. 4A), the electrostatic interaction will stretch the stalk and the charge will end up in the energetically most favourable position. We call this charge on a stalk the semi-mobile charge. For most of the positions of the anchor points, the semi-mobile charge will be attracted either to the fixed S4 charge or to the high dielectric water, independent of the stalk length l. However, for some anchor positions, the semi-mobile charge will be attracted to the fixed S4 charge (i in FIG. 4A) for short stalk lengths and to the high dielectric water (ii in FIG. 4A) for long stalk lengths. The switch from one direction to the other occurs at a specific stalk length (the cut-off length). For each position of the fixed S4 charge, the area for these anchor points can be calculated and the cut-off lengths colour coded (FIG. 4C for the positions d=2 Å, d=4 Å, d=6 Å).

Even though the distance d is not known some general conclusions can be drawn: (i) The shorter the cut-off length of the stalk is, the closer the anchor point is to the surface. (ii) For a specific cut-off length of the stalk, the possible anchor points are relatively independent of the distance d of the gating charge from the surface (FIG. 4D). Experimentally, we found that the cut-off length was roughly 4 Å. This places the anchor point about 4-6 Å from the water if the horizontal distance from the S4 charge is less than 10 Å (FIG. 4D). Having said this, we should be aware of that the quantitative estimations depend on the model and their constants.

### EXAMPLE 4

The Valence of the Charge is Critical

Above, we have analysed the effect of a single fully charged effector on stalks of different lengths. A critical finding was that, most strikingly for the wt Shaker Kv channel, there was a maximal G(V) shift when the effector group was 3 atoms away from the anchor point. However, for longer stalks, for both the wt and the 3R Shaker Kv channels, the effect was eliminated, probably because of snorkelling of the charge towards the extracellular solution.

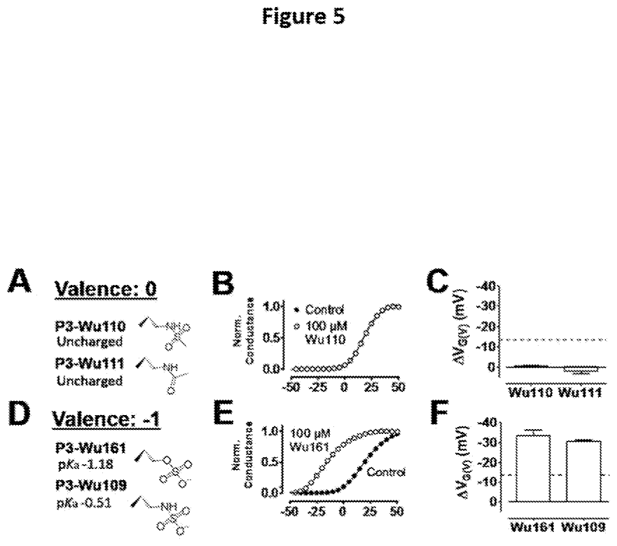

Our next hypothesis was that the G(V)-shifting effect could be increased by increasing the valance of charge on the stalk. To further corroborate that the charge was critical for the effect we introduced a neutral group instead of the carboxyl group. Two different uncharged P3 compounds (FIG. 5A, Wu110 and Wu111) tested on the 3R Shaker Kv channel at 100 μM and pH 7.4 did not shift the G(V) curve (FIG. 5B,C).

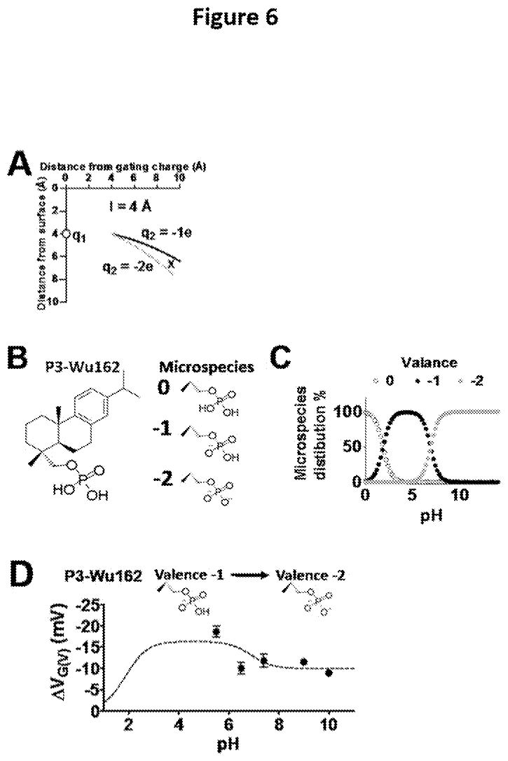

Contrary, two different permanently single-charged P3 compounds (FIG. 5D, Wu109 and Wu161) at 100 μM and pH 7.4 shifted the G(V) curve of the 3R Shaker Kv channel by approximately −30 mV (FIG. 3; FIG. 5E,F). What happens with a divalent charge? If the divalent charge is in the same position as the monovalent charge and if there is an electrostatic interaction between S4 and the compound, the effect should increase. However, our simple cut-off model also suggests another finding. At some anchor points, the double-charged group on a stalk is expected to find its way towards the water rather than to the S4 charge in the membrane, while the single-charged group on a stalk finds its way towards the S4 charge (FIG. 6A, when the semi-mobile charge is anchored (x) between the two different cut-off lines for the valences −1 and −2). Thus, it is possible that the G(V)-shifting effect also decrease with a divalent charge on the stalk.

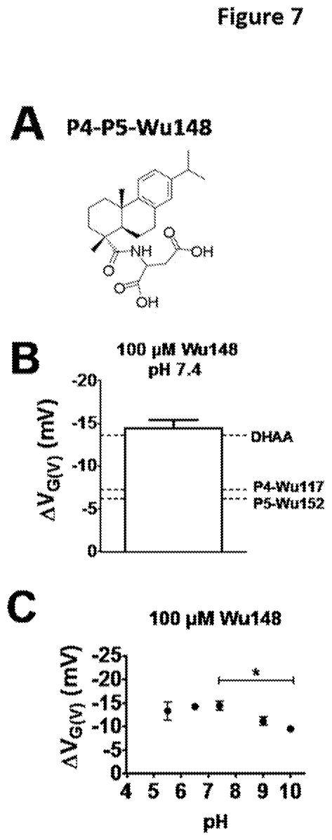

Wu162 (FIG. 6B) has a phosphorous-acid group (—P(═O)(—O⁻)₂) three atoms away from the anchor. It is a strong acid, expected to have one negative charge in the pH range from 3 to 6 (theoretically ≥90% of the molecules in this range) and with increasing pH the group is expected to have two negative charges (FIG. 6C). At pH 5.5, when nearly all Wu162 molecules are expected have a single negative charge, 100 μM Wu162 shifted the G(V) curve by almost −20 mV (FIG. 6D). The oocytes did not tolerate pH 5. When pH instead was increased above 6 the effect significantly decreased (FIG. 6D), suggesting that a stalk with a divalent charge instead finds a position further away from the voltage sensor. The dashed line is the best fit of Eq. 5. In other aspects, the divalent Wu162 molecule at pH 7.4 behaved qualitatively as the monovalent compounds: The effect was smaller on the wt Shaker Kv channel than on the 3R (Table 1). Increasing the stalk beyond four atoms decreased the voltage shift (Wu158 (P4) compared with Wu162 (P3), Table 1). Another possibility to increase the charge on the stalk was to add two monovalent charges on the same stalk, but at different positions. One P4 charge and one P5 charge (carboxyl group), sharing the first three atoms of the stalk, made it possible to add the effects from the single charged P4 and P5 molecules, but because adding two charges only is possible for longer stalks, with small effect on its own, so the sum was not very impressive (FIG. 7A-C).

In conclusion, a divalent charge does not increase the G(V)-shifting effect compared with a monovalent charge, but decreased it. A possible explanation is that a divalent charge instead finds a more energetic favourable position towards the water rather than the voltage sensor. Therefore, the stalk length is better modifier of the effect than to increase the valance above −1.

### EXAMPLE 6

Wu161—a Functional Chimera Between a Fatty Acid and a Resin Acid

The aim of this investigation was to modify the carboxyl-group of DHAA to enhance the G(V) shift. So far we have described that a three-atom long stalk combined with a decreased pKₐ value for the effector group is the most efficient modification. Increasing the valance above one did not increase the effect, but rather decreased it. P3-Wu161 (FIG. 3A) is thus one of the most intriguing compounds in this study and it holds promises for future drug-development efforts. In this section we explored the compound further, both with respect to concentration dependence and to specific channel mutations around S4 to get information about its interaction surface with the channel.

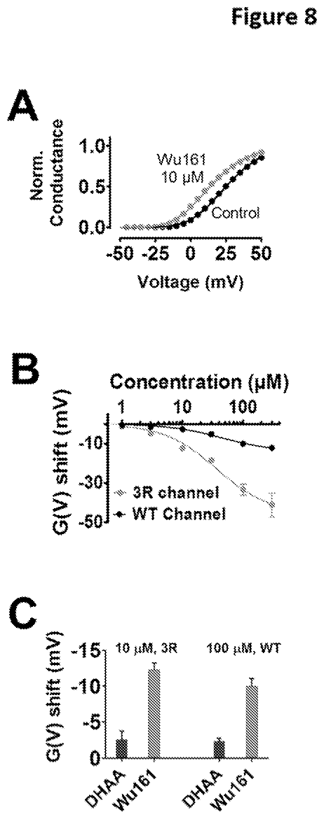

Wu161 at 10 μM, clearly shifted the G(V) of the 3R Shaker Kv channel along the voltage axis by −12.3±1.0 mV (n=4; FIG. 8A). A concentration-response curve shows a half-maximal concentration, c_{1/2}, of 36±9 μM and a maximum shift of −45.5±3.5 mV even though a saturation was not reached experimentally in the concentration range investigated (FIG. 8B, grey symbols). For the wt Shaker Kv channel the concentration dependence was not different (cy_{1/2}=44±10 μM) but the amplitude was a factor 3 smaller (−13.9±1.1 mV; FIG. 8B, black symbols). Thus, the two added charges in top of S4 (the 3R channel) does not affect the affinity of Wu161 but increases the efficacy. This 3-fold difference between the wt and the 3R Shaker Kv channel is the same as for DHAA, see Ottosson, N. E. et al. Sci. Rep. 5, 13278 (2015). However, compared to DHAA, the modification at the carboxyl-site for Wu161 (prolongation with two carbons), increased the G(V) shifting effect by a factor of about five, both on the wt and the 3R Shaker Kv channel (FIG. 8C). Data for DHAA, from Ottosson, N. E. et al. Sci. Rep. 5, 13278 (2015).

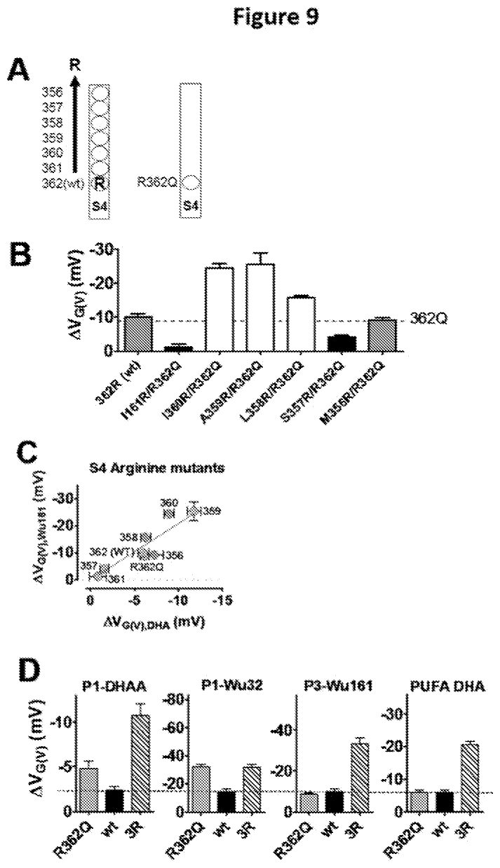

For compounds acting close to the voltage sensor, the positions of the gating charges play a large role. We explored a series of single mutations where the top charge of S4 (R362) was moved residue by residue towards the extracellular end of S4 (from R362R to M356R), or explored the effect on a channel where this area was uncharged (R362Q) (FIG. 9A). The amplitude of the effect followed an oscillatory pattern when the positive gating charge, at the top of S4, was moved along S4 (FIG. 9B). This is consistent with previous studies with the PUFA docosahexaenoic acid (DHA), see Ottosson, N. E. et al. J. Gen. Physiol. 143, 173-182 (2014) and for two P1 resin acids (DHAA, Wu32). Our data for P3-Wu161 is very similar to that of the PUFA DHA, except that Wu161 is about twice as effective, as expected from a fully charged compound (Wu161) compared with a partially charged compound (DHA). There is an almost linear relation between the effects of DHA vs. Wu161 (FIG. 9C), suggesting that DHA and Wu161 act in a similar way, probably from about the same position close to S4. In contrast with this, two P1 resin acids (DHAA and Wu32) differed from the PUFA DHA with respect to one of the channel mutations in S4: DHAA and Wu32 have an about twice as large effect on R362Q than on wt R362, while DHA and Wu161 have about the same effects on these two channels (FIG. 9D). This has been interpreted as if the P1 resin acids binds deeper into the VSD (in the S3/S4 cleft). Now, the P3 compounds seems to be more PUFA-like and we suggest that the longer stalk makes the resin acid act more a snake-like fatty acid.

### EXAMPLE 7

Combined Stalk and Anchor Modifications Enhanced the G(V) Shift

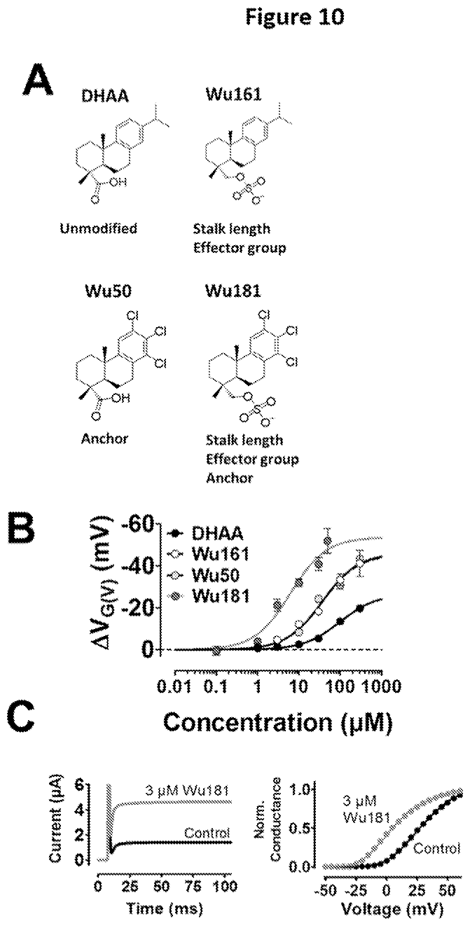

The G(V)-shifting effect of the DHAA derivatives can also be increased by modifications on the three ringed motif (the anchor), not only by modifications of the effector as shown in above. Wu161 and Wu50 (FIG. 10A) have similar effects on the 3R Shaker Kv channel (FIG. 10B), both are clearly more potent than DHAA. They both increased the affinity by a factor of 2.7 (from 98±19 μM (DHAA) to 36±9 μM (Wu50) and 37±6 μM (Wu161) respectively) and the amplitude by a factor of 1.7 (from −26.5±2.2 mV to −45.5±3.5 mV (Wu50) and −46.0±2.4 mV (for Wu161) respectively) compared to the unmodified DHAA. Data for DHAA and Wu50, from Ottosson, N. E. et al. Sci. Rep. 5, 13278 (2015). What happens if we combine an anchor modification with a stalk/effector modification? Wu181 combine both these two modifications (FIG. 10A). Wu181 has very large effects at 3 μM on the 3R Shaker Kv channel (FIG. 10C); the shift of the G(V) curve was −21.3±2.9 mV (n=5). While the max shift was not significantly increased (−53.6±4.0 mV for Wu181), the affinity was increased by a factor 6 (to 6.1±1.7 μM for Wu181). Thus, a systematic modification of the mother compound DHAA with respect to the anchor motif and the effector motif increased the affinity at pH 7.4 by a factor of 16 and the efficacy by a factor of 2.0. Altogether this suggests an increased G(V) shift at low concentrations by a factor of 32.

### EXAMPLE 8

DHAA Derivatives Open the Human M-Channel

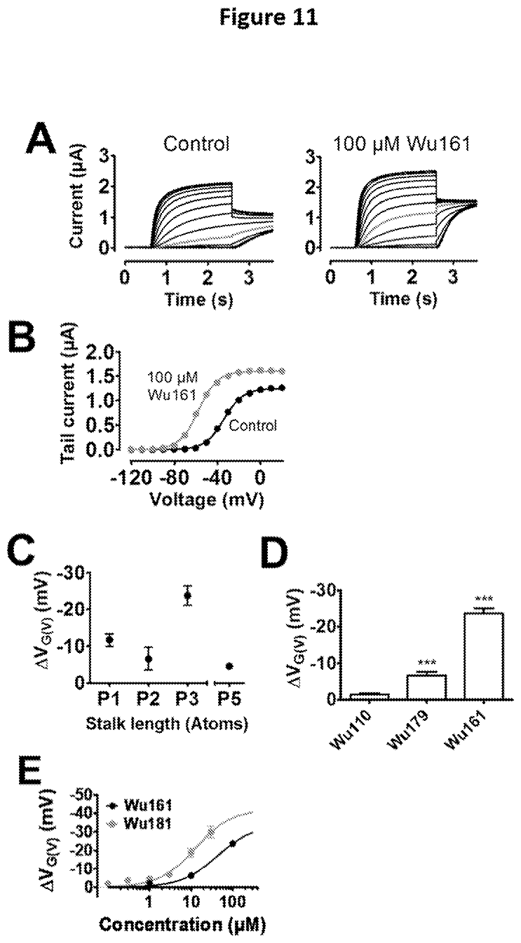

Wu161 and Wu181 were shown to have large effects on the 3R Shaker Kv channel (FIG. 11B). Wu161 functionally reminds about the PUFA DHA (FIG. 9C), which is known to act on the human M-type (hKv7.2/7.3) channel at low micromolar concentrations. Therefore, we explored the effect of Wu161 and Wu181 on the hKv7.2/7.3 channel. 100 μM Wu161 increased the M-current at negative voltages (FIG. 11A) by shifting the G(V) curve in negative direction along the voltage axis by −23.8±1.3 mV (n=4; FIG. 11B). As for the wt Shaker Kv channel a stalk length of three atoms had the largest effect on the M-channel (FIG. 11C) and the charge was also critical for the effect (FIG. 11D); the uncharged P3 compound (Wu110), had no effect, and a fully charged P3 compound (Wu161) had larger effect than a partially charged P3 compound (Wu179). 10 μM Wu161 also significantly shifted the G(V) of the M channel by −6.3±0.9 mV (n=4; FIG. 11E). The half-maximal concentration, c_{1/2}, was 42±13 μM and the maximum shift was −33.6±3.7 mV. However, Wu181, combining the effector of Wu161 with the anchor of Wu50 had a much larger effect on the M channel. 10 μM Wu181 shifted the G(V) by −18.9±2.4 mV (n=4) along the voltage axis, and 1 μM shifted the G(V) by −4.4±1.6 mV (n=4). The half-maximal concentration, c_{1/2}, was 12.4±3.7 μM and the maximum shift was −42.1±5.7 mV.

### Summary of Examples 1-8

This investigation suggests that resin acids act on a voltage-gated K channel by having (i) an anchor, which bind the molecule close to the VSD, and (ii) an effector, which electrostatically exerts the effect on the voltage sensor S4.

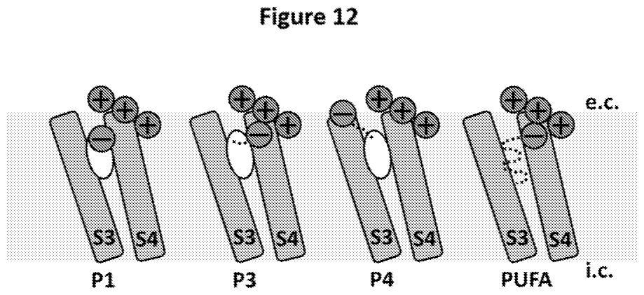

The major findings are the following:
(1) There is an optimal stalk length for the charged effector. For short stalks, the effect on voltage gating is increased when the stalk length is increased (FIG. 3E). A sudden drop in effect occurs at a certain cut-off length (FIG. 3B, 3E). These data fits with a simple electrostatic model where an increased stalk length allows the effector charge to come close to the charged voltage sensor S4 (FIG. 12, P1→P3), and that the effector charge finds a position far away from the voltage sensor when the stalk exceeds a certain cut-off length (FIG. 12, P4).
(2) The charge of the effector is absolutely critical. An uncharged molecule has no effect. But, a double charged effector does not increase the effect, but rather decrease the effect. The electrostatic model suggests that the double charged effector tends to, for electrostatic reasons, choose a location further away from the voltage sensor.
(3) The P3 molecule Wu161 affects the voltage sensor in a similar fashion as PUFAs (FIG. 9, FIG. 12).
(4) The G(V) shifting effects of an improved anchor and effector are additive, making Wu181 very potent.
(5) The human M-type Kv (hKv7.2/7.3) channel is clearly opened by 1 μM of Wu181.

Knowledge of resin acids in detail can potentially lead to development of new drugs with high specificity, affinity and selectivity.

The family of resin acids includes many compounds acting on several types of ion channels a general theme on mechanism of their effects emerges. Most compounds open voltage-gated ion channels by shifting the G(V) curve along the voltage axes. Pimaric acid, isopimaric acid, DHAA, and abietic acids shifts the G(V) of the Shaker Kv channel, while podocarpic acid with a polar side chain in its anchor does not shift the G(V). Pimaric acid shifts the voltage dependence of activation of Kv1.1-2.1 channels, but not of Kv4.3 channels. Resin acids also open ion channels outside the Kv family. They open large-conductance voltage- and Ca2+-activated K+(BK) channels, also by shifting the G(V) curve along the voltage axis. But the effects are not limited K channels but also include voltage-gated Na and Ca channels; isopimaric acid acts on five of six explored voltage-gated ion channels in a mouse cardiac atrial cell line. However, in addition to the effects on the G(V) curves, also the steady-state inactivation curves were shifted in negative direction along the voltage axis. Thus, it is clear that several resin acids act on many types of voltage-gated ion channels.

Although, binding sites for the resin acids have not been assigned to most ion channels, the communalities in effects suggests that the different types of channels share a common binding site. We have suggested that resin acids electrostatically interact with the voltage sensor of the Shaker Kv channel by binding in a pocket between the transmembrane segments S3 and S4 of the VSD, and the lipid bilayer. Together with the effects reported in the present investigation on the M channel, all these data suggests that the resin acid pocket is conserved between different Kv channels. However, it is also known that subtle side chain alterations of the compounds can have large effects on the effects, and we know that the effect varies from channel to channel. Therefore, it is likely that compounds with high channel specificity can be developed.

In the present investigation, the G(V)-shifting effect of DHAA was improved with modifications at the carboxyl-site, up to five-fold for Wu161 with a permanent negative charge on a stalk three atoms away for the anchor. Thus, modifications at the carboxyl-site can help to increase the G(V)-shifting effect and possible help to tune the resin acid sensitivity between different ion channels, with different charge profile around the voltage sensor, suggesting that the carboxyl-site of resin acids is a powerful site to modify Kv channel opening activity in future drug design.

In conclusion, all flexibilities at the carboxyl-site described in the present investigation suggests that it is likely that lipoelectric compounds can be improved to increase the G(V) shift and developed into channel specific compounds to cause desired effects in different tissues.

TABLE 1
Summary of compound properties.

| | | | | | | | | | G(V) | | |
|---|---|---|---|---|---|---|---|---|---|---|---|
| | | Position | Charged | | | | | | shift | | |
| Name | Anchor | of charge | group | LogP | pKa | Channel | pH | n | (mV) | SEM | p* |
| Wu110 | DHAA | None | None | 4.48 | — | 3R | 7.4 | 4 | 0.56 | 0.32 | N.S |
| | | | | | | M | 7.4 | 3 | —1.50 | 0.40 | N.S |
| Wu111 | DHAA | None | None | 4.99 | — | 3R | 7.4 | 3 | 1.83 | 1.25 | N.S |
| DHAA | | P1 | —COOH | 5.57 | 4.55 | 3R | 7.4 | 11 | −13.59 | 0.81 | <0.0001 |
| | | | | | | 3R | 10 | 9 | −24.73 | 1.34 | <0.0001 |
| | | | | | | wt | 10 | 7 | −1.59 | 0.35 | 0.0019 |
| | | | | | | M | 10 | 3 | −11.63 | 0.97 | 0.0069 |
| AA | | P1 | —COOH | 4.95 | 4.59 | 3R | 7.4 | 4 | −16.05 | 0.92 | 0.0004 |
| Wu180 | DHAA | P2 | —COOH | 5.68 | 4.79 | 3R | 5.5 | 4 | 1.575 | 0.47 | 0.0431 |
| | | | | | | 3R | 7.4 | 5 | −11.04 | 1.384 | 0.0013 |
| | | | | | | 3R | 10 | 5 | −19.18 | 2.739 | 0.0022 |
| | | | | | | wt | 10 | 4 | −2.40 | 0.38 | 0.0078 |
| Wu164 | DHAA | P2 | —S(═O2)OH | 4.99 | −0.49 | 3R | 7.4 | 4 | −18.00 | 0.60 | <0.0001 |
| | | | | | | wt | 7.4 | 4 | −4.10 | 0.59 | 0.0061 |
| | | | | | | M | 7.4 | 6 | −6.58 | 1.25 | 0.0033 |
| Wu179 | DHAA | P3 | —COOH | 6.13 | 4.91 | 3R | 5.5 | 5 | 2.80 | 0.45 | 0.0033 |
| | | | | | | 3R | 6.5 | 4 | −3.23 | 0.81 | N.S |
| | | | | | | 3R | 7.4 | 3 | −10.43 | 0.58 | 0.0030 |
| | | | | | | 3R | 9 | 4 | −16.63 | 0.03 | <0.0001 |
| | | | | | | 3R | 10 | 4 | −22.83 | 0.67 | <0.0001 |
| | | | | | | wt | 10 | 4 | −3.83 | 0.43 | 0.0031 |
| | | | | | | M | 7.4 | 5 | −6.68 | 1.02 | 0.0028 |
| Wu109 | DHAA | P3 | —S(═O2)OH | 4.60 | −0.51 | 3R | 7.4 | 5 | −30.68 | 0.65 | <0.0001 |
| Wu161 | DHAA | P3 | —S(═O2)OH | 5.33 | −0.18 | 3R | 5.5 | 4 | −28.10 | 1.57 | 0.0004 |
| | | | | | | 3R | 6.5 | 4 | −27.53 | 1.61 | 0.0004 |
| | | | | | | 3R | 7.5 | 6 | −33.38 | 2.77 | <0.0001 |
| | | | | | | 3R | 9 | 6 | −25.65 | 2.81 | 0.0003 |
| | | | | | | 3R | 10 | 4 | −26.38 | 1.76 | 0.0006 |
| | | | | | | wt | 7.4 | 3 | −10.00 | 1.06 | 0.0110 |
| | | | | | | M | 7.4 | 4 | −23.75 | 1.33 | 0.0004 |
| Wu181 | Wu50 | P3 | —S(═O2)OH | 5.90 | −2.04 | 3R | 7.4 | 4 | −31.90 | 3.73 | 0.0004 |
| 10 μM | | | | | | M | 7.4 | 4 | −18.90 | 2.40 | 0.0043 |
| Wu162 | DHAA | P3 | —P(═O)(—O⁻)₂ | 5.15 | 1.87/6.89 | 3R | 5.5 | 5 | ′−18.6 | 1.31 | 0.0001 |
| | | | | | | 3R | 6.5 | 4 | −10.03 | 1.40 | 0.0055 |
| | | | | | | 3R | 7.4 | 5 | −11.80 | 1.48 | 0.0013 |
| | | | | | | 3R | 9 | 4 | −11.55 | 0.25 | <0.0001 |
| | | | | | | 3R | 10 | 3 | −8.93 | 0.65 | 0.0052 |
| | | | | | | wt | 7.4 | 4 | −1.18 | 0.45 | N.S |
| Wu176 | DHAA | P3 | —COOH | 6.12 | 5.01 | 3R | 5.5 | 3 | 2.80 | 0.52 | 0.0328 |
| | | | P1=P2 | | | 3R | 7.4 | 7 | −15.16 | 1.26 | <0.0001 |
| | | | | | | 3R | 10 | 4 | −17.10 | 0.36 | <0.0001 |
| Wu117 | DHAA | P4 | —COOH | 4.64 | 4.15 | 3R | 7.4 | 6 | −7.27 | 0.36 | <0.0001 |
| | | | | | | 3R | 10 | 3 | −5.27 | 0.46 | 0.0247 |
| | | | | | | wt | 10 | 4 | −0.80 | 0.48 | N.S |
| Wu158 | DHAA | P4 | —P(═O)(—O⁻)₂ | 4.11 | 1.57/8.08 | 3R | 7.4 | 5 | −4.94 | 0.93 | 0.006 |
| Wu152 | DHAA | P5 | —COOH | 4.88 | 4.59 | 3R | 7.4 | 5 | −6.2 | 1.29 | 0.0087 |
| | | | | | | 3R | 10 | 3 | −5.20 | 0.83 | 0.0247 |
| | | | | | | wt | 10 | 3 | −1.17 | 0.26 | 0.0464 |
| Wu154 | DHAA | P5 | —S(═O2)OH | 4.19 | −0.68 | 3R | 7.4 | 4 | −9.85 | 1.352 | 0.0053 |
| | | | | | | wt | 7.4 | 5 | −2.48 | 0.20 | 0.0002 |
| | | | | | | M | 7.4 | 4 | −4.60 | 0.43 | 0.0017 |
| Wu149 | DHAA | P6 | —COOH | 5.17 | 4.32 | 3R | 7.4 | 6 | −4.80 | 0.80 | 0.0018 |
| | | | | | | 3R | 10 | 3 | −5.17 | 0.55 | 0.0111 |
| | | | | | | wt | 10 | 7 | −0.57 | 0.89 | N.S |
| Wu150 | DHAA | P6 | —S(═O2)OH | 4.25 | −0.87 | 3R | 7.4 | 4 | −9.98 | 0.63 | 0.0005 |
| | | | | | | wt | 7.4 | 4 | −4.18 | 0.44 | 0.0024 |
| Wu157 | AA | P6 | —COOH | 4.32 | 4.37 | 3R | 7.4 | 5 | −7.10 | 0.53 | 0.0002 |
| Wu153 | DHAA | P9 | —COOH | 4.01 | 4.06 | 3R | 7.4 | 5 | −2.54 | 1.36 | N.S |
| Wu151 | DHAA | P11 | —COOH | 4.59 | 4.48 | 3R | 7.4 | 4 | −2.53 | 0.42 | 0.0093 |
| Wu148 | DHAA | P4 and P5 | —COOH x2 | 6.24 | 3.88/5.71 | 3R | 5.5 | 3 | −13.30 | 2.01 | 0.0220 |
| | | | | | | 3R | 6.5 | 3 | −14.23 | 0.46 | 0.0011 |
| | | | | | | 3R | 7.4 | 6 | −14.47 | 0.97 | <0.0001 |
| | | | | | | 3R | 9 | 4 | −11.20 | 0.89 | 0.0011 |
| | | | | | | 3R | 10 | 3 | −9.53 | 0.35 | 0.0013 |
| | | | | | | wt | 7.4 | 3 | −0.73 | 0.09 | 0.0142 |

Concentration = 100 μM if not stated otherwise.
Name: Name of compound.
Anchor: Three ring structure derived from dehydroabietic acid (DHAA), abietic acid (AA) or Wu50.
Position of charge: Position of charged group as described in FIG. 2B.
Charged group: Carboxyl group —COOH, sulfonic acid group —S(═O2)OH, phosphorous-acid group (—P(═O)(—O⁻)₂).
pKₐ: calculated value for the logarithmic acid dissociation constant (see Methods).
Log P: calculated value for the logarithm of the partition coefficient (see Methods).
Channel: Channel used, wt or 3R Shaker Kv channel.
M: hKv7.2/7.3, M-channel.
pH: pH used.
Mean and SEM: average G(V) shift and standard error of mean.
n: Number of cells.

1. A dehydroabietic acid derivative according to Formula 1a or Formula 1b, or a stereoisomer thereof, wherein R₁₁, R₁₂, and R₁₄ are independently selected from hydrogen, halogen and R₂; R₁₃ is selected from hydrogen, halogen and R₃; and R₇ is selected from hydrogen, halogen, hydroxyl, carbonyl, and ═N—O—R₁; where R₁ is selected from hydrogen, and saturated or unsaturated lower alkyl groups selected from C1-C6 alkyl and C2-C6 alkenyl groups; R₂ and R₃ are independently from each other selected from straight, branched or cyclic saturated or unsaturated hydrocarbons comprising from 1 to 6 carbon atoms; wherein Formula 1a and Formula 1b are: 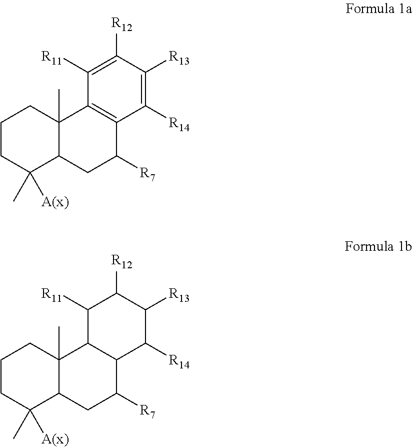
and wherein
A(x) is defined as A-X and comprises a saturated, unsaturated, branched, unbranched, substituted or unsubstituted linker chain A of 1 to 10 atoms selected from carbon, nitrogen and oxygen, between the fused tricyclic moieties of Formula 1a or 1b and at least one group X is capable of being negatively charged at a physiological pH and is selected from carboxyl, sulfate, sulfonate and phosphate groups.

2. The derivative according to claim 1, wherein R₇, R₁₁, R₁₂, and R₁₄ are hydrogen and R₁₃ is isopropyl.

3. The derivative according to claim 1, wherein the linker chain A is a carbon chain optionally interrupted by one or more atoms selected from nitrogen and oxygen and substituted with one or more of oxo groups, carboxyl groups, lower alkyl groups and halogen groups.

4. The derivative according to claim 1, wherein the linker chain A has 1 or 2 carbon atoms and X is a terminal carboxyl group.

5. The derivative according to claim 4, selected from 2-((1S,4aS)-7-isopropyl-1,4a-dimethyl-1,2,3,4,4a,9,10,10a-octahydrophenanthren-1-yl)acetic acid (Wu180) and 3-((1S,4aS)-7-isopropyl-1,4a-dimethyl-1,2,3,4,4a,9,10,10a-octahydrophenanthren-1-yl)propanoic acid (Wu179).

6. The derivative according to claim 1, wherein the linker chain A is a carbon chain, optionally interrupted with a nitrogen or oxygen atom, substituted with at least one of an oxo group and a carboxyl group, and wherein X is a terminal carboxyl group.

7. A derivative according claim 6 being ((1R,4aS,10aR)-7-isopropyl-1,4a-dimethyl-1,2,3,4,4a,9,10,10a-octahydrophenanthrene-1-carbonyl)-L-aspartic acid (Wu148).

8. The derivative according to claim 1, wherein the linker chain A is a carbon chain comprising 2 to 10 atoms of which at least one atom is nitrogen and X is a terminal carboxyl group.

9. The derivative according to claim 6, selected from group of ((1R4aS,10aR)-7-isopropyl-1,4a-dimethyl-1,2,3,4,4a,9,10,10a-octahydrophenanthrene-1-carbonyl)glycine (Wu117); 3-((1R,4aS,10aR)-7-isopropyl-1,4a-dimethyl-1,2,3,4,4a,9,10,10a-octahydrophenanthrene-1-carboxamido)propanoic acid (Wu152); 4-((1R,4aS,10aR)-7-isopropyl-1,4a-dimethyl-1,2,3,4,4a,9,10,10a-octahydrophenanthrene-1-carboxamido)butanoic acid (Wu149); 3-((1R,4aS,10aR)-7-isopropyl-1,4a-dimethyl-1,2,3,4,4a,9,10,10a-octahydrophenanthrene-1-carboxamido)propanoic acid (Wu152) and 3-(3-((1R,4aS,10aR)-7-isopropyl-1,4a-dimethyl-1,2,3,4,4a,9,10,10a-octahydrophenanthrene-1-carboxamido)propanamido)propanoic acid (Wu153); 4-(4-((1R,4aS,10aR)-7-isopropyl-1,4a-dimethyl-1,2,3,4,4a,9,10,10a-octahydrophenanthrene-1-carboxamido)butanamido)butanoic acid (Wu151); and 4-((1R,4aR,4bR,10aR)-7-isopropyl-1,4a-dimethyl-1,2,3,4,4a,4b,10,10a-decahydrophenanthrene-1-carboxamido)butanoic acid (Wu157).

10. The derivative according to claim 1, wherein the linker chain A comprises 2 to 5 atoms of which one optionally is nitrogen or oxygen and X is a terminal phosphate, sulfate or sulfonate group.

11. The derivative according to claim 10, selected from the group of ((1R,4aS,10aR)-7-isopropyl-1,4a-dimethyl-1,2,3,4,4a,9,10,10a-octahydrophenanthren-1-yl)methyl hydrogen sulfate (Wu161); 2-((1R,4aS,10aR)-7-isopropyl-1,4a-dimethyl-1,2,3,4,4a,9,10,10a-octahydrophenanthrene-1-carboxamido)ethane-1-sulfonic acid (Wu154); 3-((1R,4aS,10aR)-7-isopropyl-1,4a-dimethyl-1,2,3,4,4a,9,10,10a-octahydrophenanthrene-1-carboxamido)propane-1-sulfonic acid (Wu150); and ((1R,4aS,10aR)-7-isopropyl-1,4a-dimethyl-1,2,3,4,4a,9,10,10a-octahydrophenanthren-1-yl)methyl dihydrogen phosphate (Wu162).

12. The derivative according to claim 1, wherein R₇ is selected from hydrogen, halogen, and ═N—O—R₁ and where R₁₃ is selected from H or halogen.

13. The derivative according to claim 12, wherein the linker chain A is one carbon atom and X is sulfate.

14. (canceled)

15. The derivative according to claim 1, being ((1R,4aS,10aR)-6,7,8-trichloro-1,4a-dimethyl-2,3,4,9,10,10a-hexahydrophenanthrene-1-yl)methyl hydrogen sulfate (Wu181).

16. The derivative according to claim 2, wherein the linker chain A is a carbon chain optionally interrupted by one or more atoms selected from nitrogen and oxygen and substituted with one or more of oxo groups, carboxyl groups, lower alkyl groups and halogen groups.

17. The derivative according to claim 2, wherein the linker chain A has 1 or 2 carbon atoms and X is a terminal carboxyl group.

18. The derivative according to claim 2, wherein the linker chain A is a carbon chain, optionally interrupted with a nitrogen or oxygen atom, substituted with at least one of an oxo group and a carboxyl group, and wherein X is a terminal carboxyl group.

19. The derivative according to claim 2, wherein the linker chain A is a carbon chain comprising 2 to 10 atoms of which at least one atom is nitrogen and X is a terminal carboxyl group.

20. The derivative according to claim 2, wherein the linker chain A comprises 2 to 5 atoms of which one optionally is nitrogen or oxygen and X is a terminal phosphate, sulfate or sulfonate group.

21. A method of treating a hyperexcitability disease selected from epilepsy, pain and cardiac arrhythmia, comprising administering a derivative according to claim 1.
